(19) United States
(12) Patent Application Publication
(10) Pub. No.: US 2025/0177417 A1
(43) Pub. Date: Jun. 5, 2025

(54) COMPOSITIONS AND METHODS FOR TREATING MACULAR EDEMA ASSOCIATED WITH UVEITIS

(71) Applicant: Clearside Biomedical, Inc., Alpharetta, GA (US)

(72) Inventors: Thomas CIULLA, Alpharetta, GA (US); Shelley HANCOCK, Alpharetta, GA (US); Colette HALL, Alpharetta, GA (US); Barry KAPIK, Alpharetta, GA (US)

(21) Appl. No.: 18/726,889

(22) PCT Filed: Jan. 5, 2023
(86) PCT No.: PCT/US2023/060156

§ 371 Date: Jul. 5, 2024

Related U.S. Application Data
(60) Provisional application No. 63/297,079, filed on Jan. 6, 2022.
(60) Provisional application No. 63/298,778, filed on Jan. 12, 2022.
(60) Provisional application No. 63/301,742, filed on Jan. 21, 2022.
(60) Provisional application No. 63/359,330, filed on Jul. 8, 2022.

(51) Int. Cl. A61K 31/58; A61K 9/00; A61K 9/10; A61K 47/26; A61K 47/38; A61P 27/02

(52) U.S. Cl. CPC A61K 31/58; A61K 9/0048; A61K 9/10; A61K 47/26; A61K 47/38; A61P 27/02

(57) ABSTRACT

The present invention relates to methods, devices, and compositions for treating macular edema associated with non-infectious uveitis. For example, the methods include treatment of certain populations of subjects having macular edema associated with non-infectious uveitis by administering to the subjects a triamcinolone composition via non-surgical administration to the suprachoroidal space (SCS) of the eye.

### CROSS-REFERENCES TO RELATED APPLICATIONS

The present application claims priority and benefit of U.S. provisional application No. 63/359,330 filed Jul. 8, 2022, U.S. provisional application No. 63/301,742, filed Jan. 21, 2022, U.S. provisional application No. 63/298,778, filed Jan. 12, 2022, and U.S. provisional application No. 63/297,079, filed Jan. 6, 2022, which are incorporated herein by reference in their entireties.

### BACKGROUND OF THE INVENTION

Prolonged or severe inflammation in the back of the eye can result in the breakdown of cells at the interface of the retina and choroid, resulting in the leakage and accumulation of fluid in the macular region of the retina. This build-up of fluid can cause abnormal swelling of the macula, or macular edema, which can rapidly result in distortion of vision and eventually blindness. Because of the macula's critical role in central vision, macular edema can rapidly result in distortion of vision and eventually blindness.

Macular edema is the most frequent cause of visual impairment among patients with uveitis. Uveitis is the most common form of inflammation of the choroid and surrounding tissues in the eye, and one of the most frequent causes of blindness in the developed world. Uveitis, which can affect both eyes and is often initially diagnosed in individuals 20 to 50 years, currently accounts for 10% of vision loss/blindness in the United States and 15% worldwide, mainly occurring in the 20-50-year age group. According to studies measuring incidence and prevalence of uveitis, more than 160,000 people are diagnosed with uveitis in the United States each year. Uveitis can be infectious, meaning it is caused by an immune response to fight an infection inside the eye, or non-infectious. Non-infectious uveitis accounts for approximately 80% of all uveitis cases. Because uveitis can become chronic or recurrent if not adequately treated, some patients may become refractory, or unresponsive, to treatment, leading to irreversible blindness. Further, macular edema may persist even with successful control of the inflammatory response.

There is a need in the art for improved methods for treating uveitis, macular edema associated with uveitis, and other posterior ocular disorders. This disclosure addresses these and other needs.

### SUMMARY OF THE INVENTION

This invention is generally related to ophthalmic therapies, and more particularly to methods, devices, and compositions that allow for infusion of a drug formulation into posterior ocular tissues for targeted, localized treatment of posterior ocular disorders. For example, the compositions, devices, and methods provided herein allow for the treatment of uveitis and/or macular degeneration associated with uveitis.

In some embodiments, the present disclosure provides methods for treating macular edema associated with uveitis in a subject in need thereof, the method comprising non-surgically administering an effective amount of a triamcinolone drug formulation to the suprachoroidal space (SCS) of the eye of the human subject in need of treatment. In some embodiments, the uveitis is noninfectious uveitis. In some embodiments, the uveitis is noninfectious uveitis of posterior, intermediate, or panuveitis anatomic subtype.

In some embodiments, the effective amount of triamcinolone in the drug formulation is about 2 mg to about 5 mg. In some embodiments, the effective amount of triamcinolone in the drug formulation is about 4 mg. In some embodiments, the triamcinolone is triamcinolone acetonide. In some embodiments, the triamcinolone acetonide is formulated at 40 mg/mL. In some embodiments, the triamcinolone acetonide is formulated as a suspension of microparticles having a D₅₀ of 3 μm or less. In some embodiments, the formulation comprises 0.02% (w/v) polysorbate 80 and 0.5% (w/v) carboxymethylcellulose sodium.

In some embodiments, the uveitis is acute uveitis. In some embodiments, the uveitis is chronic uveitis. In some embodiments, the uveitis is acute uveitis. In some embodiments, the method comprises administering two doses of the triamcinolone drug formulation to the SCS of the eye of the subject. In some embodiments, the method comprises administering two or more doses of the triamcinolone drug formulation to the SCS of the eye of the subject. In some embodiments, the two or more doses of the triamcinolone drug formulation are administered about 1, 2, 3, 4, 5, 6, 7, 8, 9, 10, 11, 12, 13, 14, 15, 16, or 17 weeks apart. In some embodiments, the two doses of the triamcinolone drug formulation are administered about 12 weeks apart. In some embodiments, the two doses of the triamcinolone drug formulation are administered about 90 days apart. In some embodiments, the two doses of the triamcinolone drug formulation are administered about 12 weeks apart, and additional doses of the triamcinolone drug formulation are subsequently administered. In some embodiments, the triamcinolone drug formulation is administered to the subject by suprachoroidal injection comprising the use of an SCS microinjector. In some embodiments, the SCS microinjector comprises a 30 gauge needle that is about 900 μm or about 1100 μm in length.

In some embodiments, the subject has a visual acuity score of between about 5 and about 70 letters read before administration of the triamcinolone drug formulation.

In some embodiments, the method decreases retina thickness and/or macula thickness in a subject relative to a baseline measurement prior to treatment of the subject with the triamcinolone drug formulation. In some embodiments, the method decreases retina thickness and/or macula thickness relative to a subject that did not receive the triamcinolone drug formulation. In some embodiments, retina thickness and/or macula thickness is measured by central subfield thickness (CST). In some embodiments, the retinal thickness is decreased by at least about 20 μm, at least about 40 μm, at least about 50 μm, at least about 100 μm, at least about 125 μm, least about 150 μm, at least about 175 μm or at least about 200 μm. In some embodiments, the method results in an improvement in CST of at least 10%, at least 20%, at least 25%, at least 40%, at least 50%, at least 75%, at least 90%, or more. In some embodiments, the method resolves the increased retinal thickness in the subject. Thus, in some embodiments, the method resolves macular edema in the subject. In some embodiments, the method results in resolution of macular edema by about week 2, about week 3, about week 4, about week 5, about week 6, about week 7, or about week 8 following administration of the first dose of triamcinolone drug formulation to the SCS. In some embodiments, the resolution of macular edema in the subject is maintained for at least about 12 weeks, at least about 18 weeks, at least about 24 weeks, at least about 30 weeks, at least about 36 weeks, at least about 42 weeks, at least about 48 weeks, or longer following administration of the first dose of triamcinolone drug formulation to the SCS. In some embodiments, the method results in a statistically significant improvement in CST in a subject having posterior, intermediate, or panuveitis, relative to a subject who did not receive the triamcinolone drug formulation. In some embodiments, the method results in a statistically significant improvement in CST in a subject having posterior, intermediate, or panuveitis, relative to a subject having anterior uveitis.

In some embodiments, the method increases a visual acuity score of the subject relative to a baseline measurement prior to treatment of the subject with triamcinolone drug formulation. In some embodiments, the visual acuity score is Best Corrected Visual Acuity (BCVA). In some embodiments, the method increases the BCVA of the subject relative to a subject that did not receive the triamcinolone drug formulation. In some embodiments, the BCVA is assessed using an Early Treatment of Diabetic Retinopathy Study (ETDRS) visual acuity charts protocol. In some embodiments, the increase in the BCVA is a gain of about 5, about 6, about 7, about 8, about 9, about 10, about 12, about 14, about 15, about 16, about 17, about 18, about 19, about 20, or more letters. In some embodiments, the increase in the BCVA is a gain of at least 12 letters or 15 letters. In some embodiments, the increase in the BCVA is maintained for at least about 24 weeks, at least about 30 weeks, at least about 36 weeks, at least about 40 weeks, at least about 48 weeks, or longer following the first dose of triamcinolone drug formulation to the SCS. In some embodiments, the increase in the BCVA is maintained for at least about 24 weeks, at least about 30 weeks, at least about 36 weeks, at least about 40 weeks, at least about 48 weeks, or longer following the last dose of triamcinolone drug formulation to the SCS. In some embodiments, the method results in a statistically significant improvement in BCVA in a subject having posterior, intermediate, or panuveitis, relative to a subject who did not receive the triamcinolone drug formulation. In some embodiments, the method results in a statistically significant improvement in BCVA in a subject having posterior, intermediate, or panuveitis, relative to a subject having anterior uveitis.

In some embodiments, the method reduces inflammation in the eye of the subject. In some embodiments, the subject experiences active uveitic inflammation prior to treatment. In some embodiments, the method reduces vitreous haze, anterior chamber flare, and/or inflammatory cells in the anterior chamber. In some embodiments, the method reduces the inflammatory score in the eye of the subject by at least about 50%, at least about 75%, at least about 90%, at least about 95%, or by 100%. In some embodiments, the method resolves inflammation in the eye of the subject. In some embodiments, subjects with active inflammation at baseline (e.g., before treatment with SCS-TA) experience resolution of inflammation upon SCS-TA treatment.

In some embodiments, the triamcinolone drug formulation is administered to the subject by suprachoroidal injection comprising the use of an SCS microinjector. In further embodiments, the SCS microinjector comprises a 30 gauge needle that is about 900 μm or about 1100 μm in length.

In one aspect, the present disclosure provides methods for achieving a durable clinical outcome in a subject having macular edema associated with noninfectious posterior uveitis, noninfectious intermediate uveitis, or noninfectious panuveitis, the method comprising non-surgically administering an effective amount of a first dose of a triamcinolone drug formulation to the suprachoroidal space (SCS) of the eye of the subject. In some embodiments, the durable outcome comprises a reduction in retinal thickness in the eye of the subject. In further embodiments, the reduction in retinal thickness in the eye of the subject is a reduction in retinal thickness of at least about 20 μm, at least about 40 μm, at least about 50 μm, at least about 100 μm, at least about 150 μm, or at least about 200 μm. In some embodiments, the durable clinical outcome comprises an increase in the visual acuity score of the subject relative to a baseline measurement prior to treatment of the subject with triamcinolone drug formulation. In further embodiments, the increase in the visual acuity score is a gain of about 5, about 6, about 7, about 8, about 9, about 10, about 12, about 14, about 15, or more letters. In some embodiments, the visual acuity is measured by Best Corrected Visual Acuity (BCVA). In some embodiments, the durable clinical outcome comprises a reduction of inflammation in the eye of the subject. In some embodiments, the reduction of inflammation comprises a reduction in an inflammatory score of at least about 50%, at least about 75%, or at least about 90%, or at least about 95%. In some embodiments, the reduction of inflammation comprises a reduction in vitreous haze, anterior chamber flare, and/or inflammatory cells in the anterior chamber. In some embodiments, the durable clinical outcome is maintained for at least 24 weeks following the administration of the first dose of the triamcinolone drug formulation. In some embodiments, the durable clinical outcome is maintained for at least 48 weeks following the administration of the first dose of the triamcinolone drug formulation. In some embodiments, the uveitis is intermediate uveitis, and the durable clinical outcome is a reduction in retinal thickness of at least about 100 μm. In some embodiments, the uveitis is posterior uveitis or panuveitis, and the durable clinical outcome comprises a reduction in retinal thickness of at least about 100 μm and an increase in the visual acuity score of a gain of at least about 10 letters. In some embodiments, the method results in a statistically significant improvement in the durable clinical outcome, relative to a subject that did not receive the triamcinolone drug formulation. In some embodiments, the method results in a statistically significant improvement in the durable clinical outcome, relative to a subject having anterior uveitis.

In some embodiments, the present disclosure provides methods of treating macular edema associated with noninfectious uveitis in a subject in need thereof, comprising non-surgically administering an effective amount of a triamcinolone drug formulation to the suprachoroidal space (SCS) of the eye of the human subject in need of treatment, wherein the treatment with the triamcinolone drug formulation is initiated promptly upon diagnosis of the subject with macular edema and/or uveitis. In some embodiments, the treatment is initiated within 6 months of diagnosis of the noninfectious uveitis. In some embodiments, the treatment is initiated within 5 months, within 4 months, within 3 months, within 2 months, or within 1 month of diagnosis of the noninfectious uveitis. In some embodiments, the treatment is initiated within 6 months of diagnosis of the macular edema. In some embodiments, the treatment is initiated within 5 months, within 4 months, within 3 months, within 2 months, or within 1 month of diagnosis of the macular edema. In some embodiments, the subject has sudden disease onset prior to treatment. In some embodiments, the subject has sudden disease onset within 6 months, 5 months, 4 months, 3, months, 2 months, or 1 month prior to treatment. In some embodiments, the subject is treated as soon as possible after diagnosis of the macular edema and/or uveitis.

In some embodiments, the method results in (i) a decrease in retinal thickness relative to a baseline measurement prior to treatment of the subject with the triamcinolone drug formulation; and/or (ii) an increase in a visual acuity score of the subject relative to a baseline measurement prior to treatment of the subject with the triamcinolone drug formulation. In some embodiments, the method results in a decrease in retina thickness and/or an increase in a visual acuity score, relative to a subject that did not receive the triamcinolone drug formulation. In some embodiments, the method results in (i) a decrease in retinal thickness or (ii) an increase in a visual acuity score of the subject, relative to a subject that did not receive the triamcinolone drug formulation promptly upon diagnosis. For example, in some embodiments, the method results in (i) a decrease in retinal thickness or (ii) an increase in a visual acuity score of the subject, relative to a subject that received the triamcinolone drug formulation more than 6 months, more than 5 months, more than 4 months, or more than 3 months later than the diagnosis of the noninfectious uveitis, and/or the diagnosis of the macular edema. In some embodiments, the method results in a superior clinical effect (e.g., a larger decrease in retinal thickness and/or a larger increase in a visual acuity score) in a first subject treated more quickly after diagnosis compared to another subject that was treated longer after diagnosis compared to the first subject. In some embodiments, the method results in a superior clinical effect (e.g., a larger decrease in retinal thickness and/or a larger increase in a visual acuity score) in a first subject treated earlier in disease progression compared to another subject that was treated longer later in disease progression compared to the first subject. In some embodiments, the retinal thickness is decreased by at least about 20 μm, at least about 40 μm, at least about 50 μm, at least about 100 μm, at least about 150 μm, or at least about 200 μm. In some embodiments, the decrease in retinal thickness is maintained for at least 24, at least 36, or at least 48 weeks following the last dose of triamcinolone drug formulation to the SCS. In some embodiments, the increase in the visual acuity score is a Best Corrected Visual Acuity score gain of about 5, about 6, about 7, about 8, about 9, about 10, about 12, about 14, about 15, about 16, about 17, about 18, about 19, about 20, or more letters. In some embodiments, the increase in the visual acuity score is maintained for at least 24, at least 36, or at least 48 weeks following the last dose of triamcinolone drug formulation to the SCS.

In some embodiments, the present disclosure provides a method of treating macular edema associated with noninfectious uveitis by achieving a clinical outcome in a subject in need thereof, the method comprising non-surgically administering an effective amount of a triamcinolone drug formulation to the suprachoroidal space (SCS) of the eye of the human subject in need of treatment, wherein the human subject is 50 years old of age or older, and wherein the clinical outcome comprises an increase in BCVA visual acuity score of the subject relative to a baseline measurement prior to treatment of the subject with triamcinolone drug formulation, wherein the increase in the visual acuity score is a gain of about 5, about 6, about 7, about 8, about 9, about 10, about 12, about 14, about 15, or more letters.

### BRIEF DESCRIPTION OF THE DRAWINGS

FIGS. 1A and 1B are graphs showing the results of integrated analysis of two SCS-TA clinical studies, as described in Example 6. FIG. 1A is a graph showing mean changes in BCVA (letters) from baseline (CFB) for subjects in each arm (SCS-TA group vs. shame procedure control group). FIG. 1B provides % of patients in the SCS-TA group or control group gaining 15 or more BCVA letters from baseline at Week 4, 8, 12, 16, 20, and 24. P<0.001 vs. control for the SCS-TA group at all visits. Change in BCVA expressed as mean (SEM) and analyzed by ANOVA with treatment group and pooled country as fixed effects. % of patients with BCVA gain≥15 letters analyzed by Cochran-Mantel-Haenszel chi-square tests, with stratification by pooled country.

FIGS. 2A and 2B are graphs showing the mean changes in (FIG. 2A) CST and (FIG. 2B) proportion of patients with CST<300 μm in each arm (SCS-TA treatment group vs. sham procedure control) from Week 4 through Week 24. P<0.001 vs. control for the SCS-TA arm at all visits. Change in CST expressed as mean (SEM) and analyzed by ANOVA with treatment group and pooled country as fixed effects. % patients with CST<300 μm analyzed by Cochran-Mantel-Haenszel chi-square tests, with stratification by pooled country.

FIG. 3 is a graph showing proportion of patients with resolution of inflammation (in %) at Week 24 in each arm (SCS-TA group vs. control). Intention-to-treat population; last observation carried forward imputation. Data was analyzed by Cochran-Mantel-Haenszel chi-square tests, with stratification by pooled country.

FIG. 4 is a graph showing proportion of patients requiring rescue medication (in %) in each arm (SCS-TA group vs. control). Intention-to-treat population; last observation carried forward imputation. Data was analyzed by Cochran-Mantel-Haenszel chi-square tests, with stratification by pooled country.

FIG. 5 is a graph showing the mean change of BCVA (ETDRS letters) from baseline in chronic uveitis patients in each arm (SCS-TA group vs. control). P<0.001 vs baseline and P≤0.006 vs control at all time points. Data=mean (SEM); Intent-to-Treat Population, LOCF imputation. P-value vs baseline based on a t-test. P-value vs control based on an ANOVA with fixed effects for treatment group and pooled country.

FIG. 6 is a graph showing the mean difference in central subfield thickness (in microns) for chronic uveitis patients in each arm (SCS-TA group vs. control). P<0.001 vs baseline and P≤0.006 vs. control at all time points. Data=mean (SEM); Intent-to-Treat Population, LOCF imputation. P-value vs baseline based on a t-test. P-value vs control based on an ANOVA with fixed effects for treatment group and pooled country.

FIG. 7 is a graph showing probability of subject rescue (time to rescue) for chronic uveitis patients in each arm (SCS-TA group vs. control).

FIG. 8 is a graph showing the mean change of BCVA (ETDRS letters) from baseline in uveitis patients in each arm (SCS-TA group vs. control) in two patient groups (age ≤50 vs. age >50). P<0.001 vs baseline and P≤0.006 vs control at all time points. Data=mean (SEM); Intent-to-Treat Population, LOCF imputation. P-value vs baseline based on a t-test. P-value vs control based on an ANOVA with fixed effects for treatment group and pooled country.

FIG. 9 is a graph showing the mean difference in central subfield thickness (in microns) for uveitis patients in each arm (SCS-TA group vs. control) two patient groups (age ≤50 vs. age >50). P<0.001 vs baseline and P≤0.006 vs. control at all time points. Data=mean (SEM); Intent-to-Treat Population, LOCF imputation. P-value vs baseline based on a t-test. P-value vs control based on an ANOVA with fixed effects for treatment group and pooled country.

FIG. 10 is a graph showing probability of subject rescue for uveitis patients in each arm (SCS-TA group vs. control) in two age groups (age ≤50 vs. age >50).

FIG. 11 is a graph showing probability of new or worsening cataract adverse effects for uveitis patients in each arm (SCS-TA group vs. control) in two age groups (age ≤50 vs. age >50).

### DETAILED DESCRIPTION OF THE INVENTION

Methods, devices and drug formulations are provided herein for treating posterior ocular disorders, for example uveitis (e.g., infectious or non-infectious uveitis) and macular edema associated with uveitis. In one embodiment, the uveitis is intermediate, anterior, posterior or pan uveitis. In some embodiments, the drug formulation comprises triamcinolone. In some embodiments, the drug formulation comprises triamcinolone acetonide (TA). In some embodiments, the TA drug formulation is referred to herein as “SCS-TA.” In some embodiments, the methods comprise administering to the subject a dose of about 4 mg TA via injection in to the suprachoroidal space (SCS) of the eye of the subject. In some embodiments, the methods comprise administering to the subject two doses of about 4 mg TA via injection into the SCS of the eye of the subject. In further embodiments, the two doses are administered about 12 weeks apart.

In some embodiments, the methods comprise administering to the subject a dose of about 1 mg to 5 mg TA, for example about 1 mg, 2 mg, 3 mg, 4 mg, or 5 mg, including all values and ranges therebetween. In some embodiments, the methods comprise administering to the subject a dose of about 2 mg to 5 mg TA. In some embodiments, the methods comprise administering to the subject a dose of about 4 mg TA.

Intravitreal injections result in drugs diffusing throughout the eye, including into the lens, iris and ciliary body at the front of the eye, which for some drugs, has been associated with safety issues, such as cataracts and elevated intraocular pressure (IOP) levels. Specifically, intravitreal administration of triamcinolone (TA) has been associated with cataracts and increases in IOP levels in 20% to 60% of patients. Because SCS injection of drugs appears to result in drug remaining localized in the retina and choroid without substantial diffusion to the vitreous or the front portion of the eye, without wishing to be bound by theory, it is thought that SCS injection has the potential to reduce the incidence of these side effects.

Current treatments for ocular diseases often require intravitreal injections of anti-inflammatory or other drugs. However ocular disorders often affect the posterior segment of the eye (e.g., choroid and retina) and therefore, specific targeting of these tissues might be more beneficial in modulating disease progression.

The compositions and methods provided herein, in one embodiment, are used to restore or improve visual function. Without wishing to be bound by theory, in some embodiments the methods provided herein reduce macular edema affecting the retina, the tissue that lines the inside of the eye and is the part of the eye primarily responsible for vision, and the choroid, the layer adjacent to the retina that supplies the retina with blood, oxygen and nourishment. Macular edema is the build-up of fluid that can cause abnormal swelling of the macula, the portion of the retina responsible for central vision and color perception. This swelling can rapidly result in deterioration of vision and can eventually lead to blindness.

As used herein, “non-surgical” ocular drug delivery devices and methods refer to methods and devices for drug delivery that do not require general anesthesia and/or retrobulbar anesthesia (also referred to as a retrobulbar block). Alternatively or additionally, a “non-surgical” ocular drug delivery method is performed with an instrument having a diameter of 28 gauge or smaller (e.g., 30 gauge). Alternatively or additionally, “non-surgical” ocular drug delivery methods do not require a guidance mechanism that is typically required for ocular drug delivery via a shunt or cannula.

As used herein, “surgical” ocular drug delivery includes insertion of devices or administration of drugs by surgical means, for example, via incision to expose and provide access to regions of the eye including the posterior region, and/or via insertion of a stent, shunt, or cannula.

The surgical and non-surgical posterior ocular disorder treatment methods and devices described herein are particularly useful for the local delivery of drugs to the posterior region of the eye, for example the retinochoroidal tissue, macula, retinal pigment epithelium (RPE) and optic nerve in the posterior segment of the eye. In another embodiment, the non-surgical methods and microneedles provided herein can be used to target drug delivery to specific posterior ocular tissues or regions within the eye or in neighboring tissue. In one embodiment, the methods described herein deliver drug specifically to the sclera, the choroid, the Brach's membrane, the retinal pigment epithelium, the subretinal space, the retina, the macula, the optic disk, the optic nerve, the ciliary body, the trabecular meshwork, the aqueous humor, the vitreous humor, and/or other ocular tissue or neighboring tissue in the eye of a human subject in need of treatment. The methods and microneedles provided herein, in one embodiment, can be used to target drug delivery to specific posterior ocular tissues or regions within the eye or in neighboring tissue.

In one embodiment of the methods described herein, a patient in need of treatment is administered a drug, e.g., TA, to the suprachoroidal space of one or both eyes for at least one dosing session. Non-surgical administration, in one embodiment, is achieved by inserting a microneedle into one or both eyes of the patient, for example the sclera, and injecting or infusing a drug formulation through the inserted microneedle and into the suprachoroidal space of the eye. Surgical administration, in another embodiment, is achieved by making a conjunctival peritomy in the eye to expose and provide access to a posterior region of the eye; or by any other traditional surgical means of accessing the posterior region of the eye, known in the art. In some embodiments, the treatment is administered via a shunt, stent, or cannula that is surgically placed into the eye of the subject.

In one embodiment, the effective amount of the drug administered to the SCS provides higher therapeutic efficacy of the drug, compared to the therapeutic efficacy of the drug when the identical dosage is administered intravitreally, topically, intracamerally, parenterally or orally. In one embodiment, the microneedle drug delivery methods described herein precisely deliver the drug into the SCS for subsequent local delivery to nearby posterior ocular tissues (e.g., the retina and choroid) in need of treatment. The drug may be released into the ocular tissues from the infused volume (or, e.g., from microparticles or nanoparticles in the drug formulation) for an extended period, e.g., several hours or days or weeks or months, after the non-surgical drug administration has been completed. This beneficially can provide increased bioavailability of the drug relative, for example, to delivery by topical application of the drug formulation to ocular tissue surfaces, or increased bioavailability compared to oral, parenteral on intravitreal administration of the same drug dosage. In some embodiments, the drug formulation includes TA.

With the methods and microneedle devices described herein, the SCS drug delivery methods advantageously include precise control of the depth of insertion into the ocular tissue, so that the microneedle tip can be placed into the eye so that the drug formulation flows into the suprachoroidal space and into one or more posterior ocular tissues surrounding the SCS, e.g., the choroid and retina. In one embodiment, insertion of the microneedle is in the sclera of the eye. In one embodiment, drug flow into the SCS is accomplished without contacting underlying tissues with the microneedle, such as choroid and retina tissues.

The methods provided herein, in one embodiment, achieve delivery of drug to the suprachoroidal space, thereby allowing drug access to posterior ocular tissues (e.g., the choroid and retina) not obtainable via topical, parenteral, intracameral or intravitreal drug delivery. Because the methods provided herein deliver drug to the posterior ocular tissue for the treatment of a posterior ocular disorder, the suprachoroidal drug dose sufficient to achieve a therapeutic response and/or the frequency of dosing in a human subject treated with the methods provided herein is less than the intravitreal, topical, parenteral or oral drug dose or dosing schedule sufficient to elicit the same or substantially the same therapeutic response. In one embodiment, the SCS delivery methods described herein allow for decreased drug dose of the posterior ocular disorder treating drug, compared to the intravitreal, topical, intracameral parenteral or oral drug dose sufficient to elicit the same or substantially the same therapeutic response. In a further embodiment, the suprachoroidal drug dose sufficient to elicit a therapeutic response is 75% or less, or 50% or less, or 25% or less than the intravitreal, topical parenteral or oral drug dose sufficient to elicit a therapeutic response. The therapeutic response, in one embodiment, is a reduction in severity of a symptom/clinical manifestation of the posterior ocular disorder for which the patient is undergoing treatment, or a reduction in number of symptom(s)/clinical manifestation(s) of the posterior ocular disorder for which the patient is undergoing treatment. In some embodiments, the therapeutic response of SCS delivery methods provided herein includes the increased effectiveness and/or reduced number and/or reduced frequency of administration of other drugs to subject suffering from a posterior ocular disorder. For example, in some embodiments, the therapeutic response of the SCS delivery methods provided herein includes increased effectiveness and/or reduced number and/or reduced frequency of administration of a VEGF modulator drug.

The term “suprachoroidal space,” is used interchangeably with suprachoroidal, SCS, suprachoroid and suprachoroidia, and describes the potential space in the region of the eye disposed between the sclera and choroid. This region primarily is composed of closely packed layers of long pigmented processes derived from each of the two adjacent tissues; however, a space can develop in this region as a result of fluid or other material buildup in the suprachoroidal space and the adjacent tissues. The “supraciliary space,” as used herein, is encompassed by the SCS and refers to the most anterior portion of the SCS adjacent to the ciliary body, trabecular meshwork and limbus. Those skilled in the art will appreciate that the suprachoroidal space frequently is expanded by fluid buildup because of some disease state in the eye or as a result of some trauma or surgical intervention. In the present description, however, the fluid buildup is intentionally created by infusion of a drug formulation into the suprachoroid to create the suprachoroidal space (which is filled with drug formulation). Not wishing to be bound by theory, it is believed that the SCS region serves as a pathway for uveoscleral outflow (i.e., a natural process of the eye moving fluid from one region of the eye to the other through) and becomes a real space in instances of choroidal detachment from the sclera.

As used herein, “ocular tissue” and “eye” include both the anterior segment of the eye (i.e., the portion of the eye in front of the lens) and the posterior segment of the eye (i.e., the portion of the eye behind the lens).

As provided throughout, in one embodiment, the methods described herein are carried out with a hollow or solid microneedle, for example, a rigid microneedle. As used herein, the term “microneedle” refers to a conduit body having a base, a shaft, and a tip end suitable for insertion into the sclera and other ocular tissue and has dimensions suitable for minimally invasive insertion and drug formulation infusion as described herein. Both the “length” and “effective length” of the microneedle encompass the length of the shaft of the microneedle and the bevel height of the microneedle. In some embodiments, the microneedle used to carry out the methods described herein comprises one of the devices disclosed in U.S. Pat. No. 9,539,139, issued Jan. 10, 2017 or International Patent Application Publication No. WO2014/179698 (Application No. PCT/US2014/036590), filed May 2, 2014 and entitled “Apparatus and Method for Ocular Injection,” each of which is incorporated by reference herein in its entirety for all purposes. In some embodiments, the microneedle used to carry out the methods described herein comprises one of the devices disclosed in International Patent Application Publication No. WO2014/036009 (Application No. PCT/US2013/056863), filed Aug. 27, 2013 and entitled “Apparatus and Method for Drug Delivery Using Microneedles,” incorporated by reference herein in its entirety for all purposes. In some embodiments, the microneedle is an SCS microinjector as described herein.

In some embodiments, features of the devices, formulations, and methods are provided in U.S. Pat. No. 9,636,332, U.S. Patent Application Publication No. 2018-0042765, International Patent Application Publication Nos. WO2014/074823 (Application No. PCT/US2013/069156), WO2015/195842 (Application No. PCT/US2015/036299), WO2017/120601 (Application No. PCT/US2017/012757), and/or U.S. Patent Application Publication No. 2019/0269702, each of which is hereby incorporated by reference in its entirety for all purposes.

In one embodiment, the device used to carry out one of the methods described herein comprises the device described in U.S. Design patent application Ser. No. 29/506,275 entitled, “Medical Injector for Ocular Injection,” filed Oct. 14, 2014, the disclosure of which is incorporated herein by reference in its entirety for all purposes. In one embodiment, the device used to carry out one of the methods described herein comprises the device described in U.S. Patent Publication No. 2015/0051581 or U.S. Patent Publication No. 2017/0095339, which are each incorporated herein by reference in their entireties for all purposes. In some embodiments, such a device is an SCS microinjector as described herein.

As used herein, the terms “about” and “approximately” generally mean plus or minus 10% of the value stated. For example, about 0.5 would include 0.45 and 0.55, about 10 would include 9 to 11, about 1000 would include 900 to 1100.

As used herein, the term “hollow” includes a single, straight bore through the center of the microneedle, as well as multiple bores, bores that follow complex paths through the microneedles, multiple entry and exit points from the bore(s), and intersecting or networks of bores. That is, a hollow microneedle has a structure that includes one or more continuous pathways from the base of the microneedle to an exit point (opening) in the shaft and/or tip portion of the microneedle distal to the base.

The microneedle device in one embodiment, comprises a fluid reservoir for containing the therapeutic formulation (e.g., drug or cell formulation), e.g., as a solution or suspension, and the drug reservoir (which can include any therapeutic formulation) being in operable communication with the bore of the microneedle at a location distal to the tip end of the microneedle. The fluid reservoir may be integral with the microneedle, integral with the elongated body, or separate from both the microneedle and elongated body.

The microneedle and/or any of the components included in the embodiments described herein is/are formed and/or constructed of any suitable biocompatible material or combination of materials, including metals, glasses, semi-conductor materials, ceramics, or polymers. Examples of suitable metals include pharmaceutical grade stainless steel, gold, titanium, nickel, iron, gold, tin, chromium, copper, and alloys thereof. The polymer can be biodegradable or non-biodegradable. Examples of suitable biocompatible, biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes and copolymers and blends thereof. Representative non-biodegradable polymers include various thermoplastics or other polymeric structural materials known in the fabrication of medical devices. Examples include nylons, polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof. Biodegradable microneedles can provide an increased level of safety compared to non-biodegradable ones, such that they are essentially harmless even if inadvertently broken off into the ocular tissue.

Possible manufacturing techniques for devices suitable for use in the methods provided herein are described, for example, in U.S. Patent Application Publication No. 2006/0086689, U.S. Patent Application Publication No. 2006/0084942, U.S. Patent Application Publication No. 2005/0209565, U.S. Patent Application Publication No. 2002/0082543, U.S. Pat. Nos. 6,334,856, 6,611,707, 6,743,211 and PCT/US2014/36590, filed May 2, 2014, all of which are incorporated herein by reference in their entireties for all purposes.

In some embodiments, an apparatus includes a medicament container, a piston assembly and a handle. The medicament container defines a lumen configured to contain a medicament. A distal end portion of the medicament container includes a coupling portion configured to be removably coupled to a needle assembly. A proximal end portion of the medicament container includes a flange and a longitudinal shoulder. A distal end portion of the piston assembly includes an elastomeric member movably disposed within the lumen of the medicament container. The handle is coupled to a proximal end portion of the piston assembly such movement of the handle produces movement of the elastomeric member within the medicament container. The proximal end portion of the medicament container is movably disposed within the handle. A portion of the handle is configured to contact the flange to limit proximal movement of the handle relative to the medicament container. The handle includes a protrusion configured to engage the longitudinal shoulder of the medicament container to limit rotation of the handle relative to the medicament container.

Any of the compositions described herein can be injected using any suitable injector of the types shown and described herein. Any of the methods described herein can be performed use any suitable injector of the types shown and described herein. In this manner, the benefits of targeted drug delivery via a non-surgical approach can be realized. For example, in some embodiments, an apparatus includes a medicament container, a needle assembly, and a piston assembly. The medicament container contains a dose of a medicament, such as, for example a drug or cellular therapeutic, e.g., a steroid formulation or a cell suspension (e.g., a stem cell suspension). The dose has a delivered volume of at least about 20 μL, at least about 50 μL, at least about 100 μL, at least about 200 μL or at least about 500 μL. In one embodiment, the amount of therapeutic formulation delivered into the suprachoroidal space from the devices described herein is from about 10 μL to about 200 μL, e.g., from about 50 μL to about 150 μL. In another embodiment, from about 10 μL to about 500 μL, e.g., from about 50 μL to about 250 μL, is non-surgically administered to the suprachoroidal space.

In some embodiments, the needle or microneedle suitable for use in the methods provided herein can include a bevel or other characteristics of the types shown and described in International Patent Application Publication No. WO2014/036009 (Application No. PCT/US2013/056863), filed Aug. 27, 2013 and entitled “Apparatus and Method for Drug Delivery Using Microneedles” and/or International Patent Application Publication No. WO2014/179698 (International Application No. PCT/US2014/036590), filed May 2, 2014 and entitled “Apparatus and Method for Ocular Injection,” each of which is incorporated by reference herein in its entirety for all purposes.

The method for administration and/or additional or alternative steps are described, for example, in U.S. Patent Application Publication No. 2018-0042765 and International Patent Application Publication No. WO2015/195842 (Application No. PCT/US2015/036299), and in U.S. Patent Application Publication No. 2019-0269702.

The microneedle device for non-surgically delivering drug to the suprachoroidal space of the eye of a human subject, in one embodiment, comprises a hollow microneedle. The device may include an elongated housing for holding the proximal end of the microneedle. The device may further include a means for conducting a drug formulation through the microneedle. For example, the means may be a flexible or rigid conduit in fluid connection with the base or proximal end of the microneedle. The means may also include a pump or other devices for creating a pressure gradient for inducing fluid flow through the device. The conduit may in operable connection with a source of the drug formulation. The source may be any suitable container. In one embodiment, the source may be in the form of a conventional syringe. The source may be a disposable unit dose container.

The microneedle, in one embodiment, is part of an array of two or more microneedles such that the method further includes inserting at least a second microneedle into the sclera without penetrating across the sclera. In one embodiment, where an array of two or more microneedles are inserted into the ocular tissue, the drug formulation of each of the two or more microneedles may be identical to or different from one another, in drug, formulation, volume/quantity of drug formulation, or a combination of these parameters. In one case, different types of drug formulations may be injected via the one or more microneedles. For example, inserting a second hollow microneedle comprising a second drug formulation into the ocular tissue will result in delivery of the second drug formulation into the ocular tissue.

In some embodiments, a microneedle has an effective length of between about 200 μm and about 1500 μm. A short effective length microneedle (e.g., a length of between about 200 μm and about 400 μm) can be used, for example, in various subdermal injection procedures. Injectors with a longer effective length microneedle (e.g., a length of between about 600 μm and about 1500 μm) can be used, for example, in various ocular procedures, such as, injection into the subretinal space. For example, the microneedle has an effective length of between about 600 μm and about 800 μm, about 700 μm and about 900 μm, about 800 μm and about 1000 μm, about 900 μm and about 1100 μm, about 1000 μm and about 1200 μm, about 1100 μm and about 1300 μm, about 1200 μm and about 1400 μm, or about 1300 μm and about 1500 μm, including any values and ranges therebetween. In some embodiments, the microneedle has an effective length of about 600 μm, about 700 μm, about 800 μm, about 900 μm, about 1000 μm, about 1100 μm, about 1200 μm, about 1300 μm, about 1400 μm, or about 1500 μm, including any values and ranges therebetween. In some embodiments, the microneedle has an effective length of about 900 μm. In some embodiments, the microneedle has an effective length of about 1100 μm.

The SCS drug delivery methods provided herein allow for the delivery of drug formulation over a larger tissue area and to more difficult to target tissue in a single administration as compared to previously known needle devices. Not wishing to be bound by theory, it is believed that upon entering the SCS the drug formulation flows circumferentially from the insertion site toward the retinochoroidal tissue, macula, and optic nerve in the posterior segment of the eye as well as anteriorly toward the uvea and ciliary body. In addition, a portion of the infused drug formulation may remain in the SCS as a depot, or remain in tissue overlying the SCS, for example the sclera, near the microneedle insertion site, serving as additional depot of the drug formulation that subsequently can diffuse into the SCS and into other adjacent posterior tissues.

The terms “subject” and “patient” are used interchangeably herein. The human subject treated with the methods and devices provided herein may be an adult or a child. In some embodiments, the subject is at least 40 years old, 45 years old, 50 years old, 55 years old, 60 years old, 65 years old, or 70 years old, including any values or ranges therebetween. In one embodiment, the patient presents with a retinal thickness of greater than 300 μm (e.g., central retinal thickness or central subfield thickness as measured by optical coherence tomography). In another embodiment, the patient in need of treatment has a BCVA score of ≥20 letters read in each eye (e.g., 20/400 Snellen approximate). In yet another embodiment, the patient in need of treatment has a BCVA score of ≥20 letters read in each eye (e.g., 20/400 Snellen approximate), but ≤70 letters read in the eye in need of treatment.

The patient in some embodiments has macular edema (ME). The patient in one embodiment has macular edema (ME) that involves the fovea. In one embodiment, in a method for treating ME associated with uveitis, the ME is due to the uveitis and not due to any other cause. In one embodiment, the patient in need of treatment experiences a decrease in visual acuity due to the ME.

The microneedle devices and non-surgical methods described herein may be used to deliver drug formulations to the eye of a human subject, particularly for the treatment, diagnosis, or prevention of a posterior ocular disorder, such as uveitis (e.g., non-infectious, infectious, intermediate, anterior, posterior or pan uveitis), macular edema associated with uveitis, e.g., non-infectious uveitis, including non-infectious uveitis of any anatomical subtype, e.g., intermediate, anterior, posterior or pan uveitis. In one embodiment, the drug formulation comprises an effective amount of an anti-inflammatory drug. In one embodiment, the patient is in need of treatment of macular edema associated with uveitis and the drug formulation comprises an anti-inflammatory drug selected from a steroid compound and a non-steroidal anti-inflammatory drug (NSAID). In even a further embodiment, the drug formulation is a triamcinolone formulation, e.g., a triamcinolone acetonide formulation.

The uveitis can be either acute or chronic uveitis. Uveitis, and macular edema associated with uveitis can be caused by infectious causes leading to infectious uveitis, such as infection with viruses, fungi, parasites, and/or the like. Uveitis can also be caused by non-infectious causes, such as the presence of noninfectious foreign substances in the eye, autoimmune diseases, surgical and/or traumatic injury, and/or the like. Disorders caused by pathogenic organisms that can lead to infectious uveitis, and to macular edema associated with infectious uveitis, include, but are not limited to, toxoplasmosis, toxocariasis, histoplasmosis, herpes simplex or herpes zoster infection, tuberculosis, syphilis, sarcoidosis, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, or iridocyclitis. Acute uveitis and/or macular edema associated with acute uveitis occurs suddenly and may last for up to about six weeks. In chronic uveitis and/or macular edema associated with chronic uveitis, the onset of signs and/or symptoms is gradual, and symptoms last longer than about six weeks. In some embodiments, chronic uveitis is persistent uveitis. In some embodiments, chronic uveitis is persistent uveitis with relapse in <3 months after discontinuing prior treatment. The uveitis can be of any anatomic subtype (anterior, intermediate, posterior, or panuveitis).

Signs of uveitis include ciliary injection, aqueous flare, the accumulation of cells visible on ophthalmic examination, such as aqueous cells, retrolental cells, and vitreous cells, keratic precipitates, and hypema. Symptoms of uveitis include pain (such as ciliary spasm), redness, photophobia, increased lacrimation, and decreased vision. Posterior uveitis affects the posterior or choroid part of the eye. Inflammation of the choroid part of the eye is also often referred to as choroiditis. Posterior uveitis is may also be associated with inflammation that occurs in the retina (retinitis) or in the blood vessels in the posterior segment of the eye (vasculitis). In one embodiment, the methods provided herein comprise non-surgically administering to a uveitis patient suffering from macular edema associated with uveitis (e.g., non-infectious uveitis) in need thereof, an effective amount of an anti-inflammatory drug formulation to the SCS of the eye of the patient. In a further embodiment, the patient experiences a reduction in the severity of the symptoms of with macular edema associated with uveitis, after administration of the drug formulation.

In one embodiment, the patient undergoing one of the treatment methods provided herein, for example, the treatment of macular edema associated with uveitis, experiences a reduction in fluid accumulation, inflammation, neuroprotection, complement inhibition, drusen formation, scar formation, and/or a reduction in choriocapillaris or choroidal neovascularization. Without wishing to be bound by theory, upon non-surgical SCS administration, the drug remains localized in the posterior segment of the eye, specifically, the choroid and retina. Limiting drug exposure to other eye tissues, in one embodiment, reduces the incidences of side effects associated with the prior art methods.

In one embodiment, from about 2 to about 24 dosing sessions are employed, for example, from about 2 to about 24 intraocular dosing sessions (e.g., intravitreal or suprachoroidal injection). In a further embodiment, from about 3 to about 30, or from about 5 to about 30, or from about 7 to about 30, or from about 9 to about 30, or from about 10 to about 30, or from about 12 to about 30 or from about 12 to about 24 dosing sessions are employed. In particular embodiments, the methods provided herein comprise two dosing sessions, wherein 4 mg TA is administered via non-surgical administration to the SCS in each of the two dosing sessions. In some embodiments, the two dosing sessions are about 1 2, 3, 4, 5, 6, 7, 8, 9, 10, 11, 12, 13, 14, 15, 16, 17, 18, 19, 20, 21, 22, 23, or 24 weeks apart. In certain embodiments, the two dosing sessions are about 12 weeks apart.

Treatment regimens will vary based on the therapeutic formulation being delivered and/or the indication being treated. In one embodiment, a single dosing session is effective in treating one of the indications described herein. However, in another embodiment, multiple dosing sessions are employed. In one embodiment, where multiple dosing sessions are employed, the dosing sessions are spaced apart by from about 10 days to about 100 days, or from about 10 days to about 90 days, or from about 10 days to about 80 days, or from about 10 days to about 40 days, or from about 10 days to about 30 days, or from about 10 days to about 20 days. In another embodiment, where multiple dosing sessions are employed, the dosing sessions are spaced apart by from about 20 days to about 60 days, or from about 20 days to about 50 days, or from about 20 days to about 40 days, or from about 20 days to about 30 days. In another embodiment, the dosing sessions are spaced apart by about 42 days, about 39 days, about 56 days, about 63 days, about 70 days, about 77 days, about 84 days, or about 91 days, about 98 days. In some embodiments, the dosing sessions are spaced apart by about 84 days. In even another embodiment, the multiple dosing sessions are weekly (about every 7 days), bi-weekly (e.g., about every 14 days), about every 21 days, monthly (e.g., about every 30 days), bi-monthly (e.g., about every 60 days), or every three months. In yet another embodiment, the dosing sessions are monthly dosing sessions (e.g., from about 28 days to about 31 days) and at least three dosing sessions are employed.

In one embodiment, the treatment methods provided herein (e.g., non-surgical SCS delivery of a triamcinolone acetonide formulation) are initiated promptly after diagnosis of the subject with the disorder to be treated. For example, in an embodiment, the treatment method is initiated within 1 week, within 2 weeks, within 3 weeks, within 1 month, within 2 months, within 3 months, within 4 months, within 5 months, or within 6 months, of diagnosis with noninfectious uveitis and/or with macular edema. For example, in an embodiment, the treatment method is initiated within 30, 40, 50, 60, 70, 80, 90, 100, 110, 120, 130, 140 150, 160, 170, or 180 days of diagnosis with noninfectious uveitis and/or with macular edema. In some embodiments, the subject has had a duration of disease of less than 6 months, less than 5 months, less than 4 months, or less than 3 months prior to initiation of treatment. In some embodiments, the subject treated with the treatment methods provided herein exhibits or was diagnosed with sudden onset disease, as opposed to insidious onset disease. In some embodiments, the treatment method provides a superior clinical outcome in patients who are treated earlier in the course of disease relative to patients treated later. In some embodiments, the treatment method provides a superior clinical outcome in patients who are treated closer to the time of diagnosis, relative to patients treated a longer time after diagnosis.

In one embodiment, the non-surgical SCS delivery methods, for example, with one of the devices provided herein, are used to treat a patient in need of treatment of macular edema associated with uveitis (e.g., non-infectious uveitis). In one embodiment, SCS administration of a drug (e.g., an anti-inflammatory compound such as a steroid or NSAID) via the methods described herein reduces the vitreous haze experienced by the patient.

In one embodiment, vitreous haze is assessed via indirect ophthalmoscopy using a standardized photographic scale ranging from 0 to 4, with 0-4 defined below in Table 1 (Nussenblatt 1985 as modified in Lowder 2011, incorporated by reference herein in their entireties). Vitreous haze in another embodiment, is graded from color fundus photographs according to a similar scale.

TABLE 1

| Score | Description |
|---|---|
| 0 | No inflammation |
| +0.5 | Trace inflammation (slight blurring of the optic disc margins |
| | and/or loss of the nerve fiber layer reflex) |
| +1 | Mild blurring of the retinal vessels and optic nerve |
| +1.5 | Optic nerve head and posterior retina view obsuration greater |
| | than +1 but less than +2 |
| +2 | Moderate blurring of the optic nerve head |
| +3 | Marked blurring of the optic nerve head |
| +4 | Optic nerve head not visible |

The efficacy of the method, in one embodiment, is measured by measuring the patient's mean change from baseline in macula thickness at one or more time points after the patient is treated. For example, at one week, two weeks, three weeks, one month, two months, three months, four months, five months, six months, or more, including all durations in between, after treatment, e.g., with an anti-inflammatory drug delivered non-surgically to the SCS, mean change from baseline in retinal thickness and/or macula thickness is measured.

A decrease in retina thickness and/or macula thickness is one measurement of treatment efficacy of the methods provided herein. For example, in one embodiment, a patient treated by one of the methods provided herein for example with one of the devices described herein experiences a decrease in retinal thickness from baseline (e.g., retinal thickness such as central retinal thickness (CRT) or central subfield thickness (CST) prior to treatment), at any given time point after at least one dosing session (single session or multiple dosing sessions), of at least about 20 μm, or at least about 40 μm, or at least about 50 μm, or at least about 100 μm, or at least about 150 μm or at least about 200 μm, or from about 50-100 μm, or from about 75-200 μm, or from about 100-150 μm, or from about 150-200 μm, and all values in between. In another embodiment, the patient experiences a ≥5%, ≥10%, ≥15%, ≥20%, ≥25%, ≥25%, ≥30%, ≥35%, or ≥40%, decrease in retinal thickness (e.g., CST) subsequent to at least one dosing session.

In one embodiment, the decrease in retinal thickness is measured about 2 weeks, about 1 month, about 2 months, about 3 months, about 6 months, or longer after the at least one dosing session. In another embodiment, the decrease in retinal thickness is measured at least about 2 weeks, at least about 1 month, at least about 2 months, at least about 3 months, at least about 6 months, or longer after the at least one dosing session. In one embodiment, where multiple dosing sessions are employed, a decrease in retinal thickness is sustained by the patient for at least about 2 weeks, at least about 1 month, at least about 2 months, at least about 3 months, at least about 6 months, or longer after each dosing session.

In one embodiment, a macular edema associated with uveitis (e.g., non-infectious uveitis) patient treated by the methods provided herein experiences a decrease in retinal thickness from baseline (i.e., retinal thickness prior to treatment), at any given time point, of from about 20 μm to about 200 μm, at from about 40 μm to about 200 μm, of from about 50 μm to about 200 μm, of from about 100 μm to about 200 μm, or from about 150 μm to about 200 μm. In one embodiment, change in retinal thickness from baseline is measured as a change in CRT or CST, for example, by spectral domain optical coherence tomography (SD-OCT).

In yet another embodiment, the therapeutic response is a change from baseline in macula thickness at one or more time points after the patient is treated. For example, at one week, two weeks, three weeks, one month, two months, three months, four months or more, including all durations in between, after a dosing session, e.g., with an anti-inflammatory drug such as triamcinolone delivered non-surgically to the SCS, change from baseline in macula thickness is measured. A decrease in macula thickness (as compared to prior to treatment) is one measurement of therapeutic response (e.g., by about 10%, or about 20%, or about 30%, or about 40%, or about 50%, or about 60% and more, including all values in between).

Efficacy, in another embodiment, is assessed via a visual acuity measurement at one and/or two months post treatment (e.g., by measuring the mean change in best corrected visual acuity (BCVA) from baseline, i.e., prior to treatment). In one embodiment, a patient treated by one or more of the methods provided herein experiences an improvement in BCVA from baseline, at any given time point (e.g., 2 weeks after administration, 4 weeks after administration, 2 months after at least one dosing session, 3 months after administration), of at least 2 letters, at least 3 letters, at least 5 letters, at least 8 letters, at least 12 letters, at least 13 letters, at least 15 letters, at least 20 letters, and all values in between, as compared to the patient's BVCA prior to the at least one dosing session.

In one embodiment, the patient gains about 5 letters or more, about 10 letters or more, about 15 letters or more, about 20 letters or more, about 25 letters or more in a BCVA measurement after a dosing regimen is complete, for example a monthly dosing regimen, compared to the patient's BCVA measurement prior to undergoing treatment. In even a further embodiment, the patient gains from about 5 to about 30 letters, 10 to about 30 letters, from about 15 letters to about 25 letters or from about 15 letters to about 20 letters in a BCVA measurement upon completion of at least one dosing session, compared to the patient's BCVA measurement prior to the at least one dosing session. In one embodiment, the BCVA gain is about 2 weeks, about 1 month, about 2 months, about 3 months or about 6 months after the at least one dosing session. In another embodiment, the BCVA is measured at least about 2 weeks, at least about 1 month, at least about 2 months, at least about 3 months or at least about 6 months after the at least one dosing session.

In one embodiment, the BCVA is based on the Early Treatment of Diabetic Retinopathy Study (ETDRS) visual acuity charts.

In another embodiment, the patient subjected to a treatment method, e.g., with one of the devices provided herein substantially maintains his or her vision subsequent to the treatment (e.g., a single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

Decrease in vitreous haze can also be used as a measure of the method's efficacy. Decreases in vitreous haze can be qualitatively and/or quantitatively determined by techniques such as, but not limited to, photographic grading, a scoring system, a multi-point scale, a multi-step scale (e.g. a multi-step logarithmic scale, manual screening by one or more examiners, and/or the like).

In one embodiment, the decrease in vitreous haze is present about 2 weeks, about 1 month, about 2 months, about 3 months or about 6 months after the at least one dosing session. In another embodiment, the decrease in retinal thickness is present at least about 2 weeks, at least about 1 month, at least about 2 months, at least about 3 months or at least about 6 months after the at least one dosing session. In one embodiment, where multiple dosing sessions are employed, a decrease in vitreous haze is experienced by the patient and is present at least about 2 weeks, at least about 1 month, at least about 2 months, at least about 3 months or at least about 6 months after each dosing session.

In a further embodiment, a second drug formulation comprising a VEGF modulator (e.g., a VEGF antagonist) is administered to the eye of the patient via an intravitreal injection. In a further embodiment, the VEGF modulator is ranibizumab, aflibercept or bevacizumab. In some embodiments, the methods provided herein include methods for treating diabetic macular edema (DME) in a patient in need thereof, the method comprising administering an effective amount of a triamcinolone drug formulation to the SCS of the eye of the patient, and further comprising administering a VEGF modulator to the eye of the patient. In further embodiments, the VEGF modulator is administered to the eye via intravitreal injection. In other embodiments, the VEGF modulator is administered to the eye via SCS administration. In some embodiments, the VEGF modulator is aflibercept.

In one embodiment, the methods provided herein provide for effective treatment of a patient who had previously undergone treatment for a posterior ocular disorder, but was unresponsive, or not properly responsive to the prior treatment for the respective posterior ocular disorder. As one of skill in the art will appreciate, a patient unresponsive or not properly responsive to treatment does not exhibit an improvement in a symptom or improvement in a clinical manifestation of macular edema associated with the disorder. In one embodiment, the symptom or clinical manifestation is lesion size, inflammation, edema, visual acuity and/or vitreous haze.

In patients undergoing ocular treatment via shunts or cannulae, or other surgical methods, a marked increase or decrease in intraocular pressure has been reported after the treatment method commences. In one embodiment, the intraocular pressure (IOP) of the patient's eye undergoing treatment for uveitis or macular edema associated with uveitis (e.g., non-infectious uveitis), 2 minutes, 10 minutes, 15 minutes, 30 minutes or 1 hour after suprachoroidal drug administration according to the devices (e.g., the device 100) and/or the methods disclosed herein, is substantially the same IOP, compared to the TOP of the patient's eye prior to administration of the drug. In one embodiment, the IOP of the patient's eye undergoing treatment for uveitis or macular edema associated with uveitis (e.g., non-infectious uveitis), 2 minutes, 10 minutes, 15 minutes, 30 minutes or 1 hour after suprachoroidal drug administration, varies by no more than 10%, compared to the TOP of the patient's eye prior to administration of the drug. In one embodiment, the IOP of the patient's eye undergoing treatment for the uveitis or macular edema associated with uveitis (e.g., non-infectious uveitis) 2 minutes, 10 minutes, 15 minutes or 30 minutes after suprachoroidal drug administration, varies by no more than 20%, compared to the IOP of the patient's eye prior to administration of the drug. In one embodiment, the IOP of the patient's eye undergoing treatment for uveitis or macular edema associated with uveitis (e.g., non-infectious uveitis), 2 minutes, 10 minutes, 15 minutes or 30 minutes after suprachoroidal drug administration, varies by no more than 10%-30%, compared to the TOP of the patient's eye prior to administration of the drug. In a further embodiment, the effective amount of the drug for treating uveitis or macular edema associated with uveitis (e.g., non-infectious uveitis), comprises an effective amount of an anti-inflammatory drug (e.g., triamcinolone).

In one aspect, the methods described herein relate to the administration of a drug formulation for the treatment of uveitis (infectious or non-infectious), macular edema, macular edema associated with non-infectious uveitis, macular edema associated with infectious uveitis, wherein the majority of the drug formulation is retained in the SCS and/or other posterior ocular tissue, in one or both eyes of a patient in need of treatment of the posterior ocular disorder, for a period of time after the treatment method is completed. Without wishing to be bound by theory, drug formulation retention in the SCS contributes to the sustained release profile of the drug formulations described herein.

In one embodiment, the methods provided herein allow for greater drug retention in the eye compared to other drug delivery methods, for example, a greater amount of drug is retained in the eye when delivered via the methods provided herein as compared to the same dose delivered via intracameral, sub-tenon, intravitreal, topical, parenteral or oral drug delivery methods. Accordingly, in one embodiment, the intraocular elimination half life (t_{1/2}) of the drug when delivered via the methods described herein is greater than the intraocular t_{1/2} of the drug when the same drug dose is administered intravitreally, intracamerally, topically, parenterally or orally. In a further embodiment, the intraocular t_{1/2} of the drug when administered via the non-surgical SCS drug delivery methods provided herein, is from about 1.1 times to about 10 times longer, or from about 1.25 times to about 10 times longer, or from about 1.5 times to about 10 times longer, or about 2 times to about 5 times longer, than the intraocular t_{1/2} of the drug when the identical dosage is administered topically, intracamerally, sub-tenonally, intravitreally, orally or parenterally. In another embodiment, the intraocular Cmax of the drug, when delivered via the methods described herein, is greater than the intraocular Cmax of the drug when the same drug dose is administered intravitreally, intracamerally, sub-tenonally, topically, parenterally or orally. In a further embodiment, the intraocular Cmax of the drug when administered via the non-surgical SCS drug delivery methods provided herein, is at least 1.1 times greater, or at least 1.25 times greater, or at least 1.5 times greater, or at least 2 times greater, or at least 5 times greater, than the intraocular Cmax of the drug when the identical dose is administered topically, intracamerally, intravitreally, orally or parenterally. In one embodiment, the intraocular Cmax of the drug when administered via the non-surgical SCS drug delivery methods provided herein, is about 1 to about 2 times greater, or about 1.25 to about 2 times greater, or about 1 to about 5 times greater, or about 1 to about 10 times greater, or about 2 to about 5 times greater, or about 2 to about 10 times greater, than the intraocular Cmax of the drug when the identical dose is administered topically, intracamerally, sub-tenonally, intravitreally, orally or parenterally. In another embodiment, the mean intraocular area under the curve (AUC₀₋ₜ) of the drug, when administered to the SCS via the methods described herein, is greater than the intraocular AUC₀₋ₜ of the drug, when administered intravitreally, intracamerally, sub-tenonally, topically, parenterally or orally. In a further embodiment, the intraocular AUC₀₋ₜ of the drug when administered via the non-surgical SCS drug delivery methods provided herein, is at least 1.1 times greater, or at least 1.25 times greater, or at least 1.5 times greater, or at least 2 times greater, or at least 5 times greater, than the intraocular AUC₀₋ₜ of the drug when the identical dose is administered topically, intracamerally, sub-tenonally, intravitreally, orally or parenterally. In one embodiment, the intraocular AUC₀₋ₜ of the drug when administered via the non-surgical SCS drug delivery methods provided herein, is about 1 to about 2 times greater, or about 1.25 to about 2 times greater, or about 1 to about 5 times greater, or about 1 to about 10 times greater, or about 2 to about 5 times greater, or about 2 to about 10 times greater, than the intraocular AUC₀₋ₜ of the drug when the identical dose is administered topically, intracamerally, sub-tenonally, intravitreally, orally or parenterally. In yet another embodiment, the intraocular time to peak concentration (tmax) of the drug, when administered to the SCS via the methods described herein, is greater than the intraocular tmax of the drug, when the same drug dose is administered intravitreally, intracamerally, topically, parenterally or orally.

In one embodiment, the drug formulation comprising the effective amount of the drug (e.g., an anti-inflammatory drug (e.g., a steroid such as triamcinolone or NSAID), once delivered to the SCS, is substantially retained in the SCS over a period of time. For example, in one embodiment, about 80% of the drug formulation is retained in the SCS for about 30 minutes, or about 1 hour, or about 4 hours or about 24 hours or about 48 hours or about 72 hours. In this regard, a depot of drug is formed in the SCS and/or surrounding tissue, to allow for sustained release of the drug over a period of time.

In one embodiment, the suprachoroidal drug delivery methods provided herein result in an increased therapeutic efficacy and/or improved therapeutic response, as compared to oral, parenteral, sub-tenon, and/or intravitreal drug delivery methods of the identical or similar drug dose. In one embodiment, the SCS drug dose sufficient to provide a therapeutic response is about 90%, or about 75%, or about one-half (e.g., about one half or less) the intravitreal, intracameral, topical, oral or parenteral drug dose sufficient to provide the same or substantially the same therapeutic response. In another embodiment, the SCS dose sufficient to provide a therapeutic response is about one-fourth the intravitreal, intracameral, sub-tenon, topical, oral or parenteral drug dose sufficient to provide the same or substantially the same therapeutic response. In yet another embodiment, the SCS dose sufficient to provide a therapeutic response is one-tenth the intravitreal, intracameral, sub-tenon, topical, oral or parenteral drug dose sufficient to provide the same or substantially the same therapeutic response. In one embodiment, the therapeutic response is a decrease in inflammation, as measured by methods known to those of skill in the art. In another embodiment, the therapeutic response is a decrease in number of ocular lesions, or decrease in ocular lesion size. In another embodiment, the therapeutic response is a decrease in fluid accumulation and/or intraocular pressure.

In one embodiment, the amount of therapeutic formulation delivered into the suprachoroidal space from the devices described herein is from about 10 μL to about 200 μL, e.g., from about 50 μL to about 150 μL. In another embodiment, from about 10 μL to about 500 μL, e.g., from about 50 μL to about 250 μL, is non-surgically administered to the suprachoroidal space.

Therapeutic response is measured at a time point post-treatment, for example 5 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks 24 weeks, 30 weeks, 36 weeks, 42 weeks, 48 weeks, or longer post-treatment, and all values in between.

The therapeutic efficacy of the drug formulations delivered by the methods described herein and therapeutic response of the human subject can be assayed by standard means in the art, as known to those of skill in the art. In general, the therapeutic efficacy of any particular drug can be assessed by measuring the response of the human subject after administration of the drug; a drug with a high therapeutic efficacy will show a greater amelioration and/or discontinuation of symptoms than a drug with a lower therapeutic efficacy. In non-limiting examples, the efficacy of the drug formulations provided herein can be measured, for example, by observing changes in pain intensity, changes in ocular lesions (size or number), intraocular pressure, fluid accumulation, inflammation (e.g., by measuring changes in the Hackett/McDonald ocular score), ocular hypertension, and/or visual acuity.

In another embodiment, the efficacy of the therapeutic formulation is measured by observing changes in the measurements according to the Hackett/McDonald ocular scores, inflammation, visual acuity, and/or edema. In another embodiment, the efficacy of the therapeutic formulation is measured, for example, by observing changes in the measurements according to the Hackett/McDonald ocular scores, inflammation, visual acuity, and/or edema.

In one embodiment, the non-surgical administration of an effective amount of a drug formulation to the SCS results to treat macular edema associated with uveitis results in a decreased number of deleterious side effects or clinical manifestations in the treated patient as compared to the number of side effects or clinical manifestations caused by the same drug dose administered intravitreally, intracamerally, orally or parenterally. In another embodiment, the non-surgical administration of an effective amount of a drug formulation to the SCS results in a decreased number of one or more deleterious side effects or clinical manifestations, as compared to the deleterious side effects or clinical manifestations caused by the same drug dose administered intravitreally, intracamerally, sub-tenonally, orally or parenterally.

Examples of side effects and clinical manifestations that can be reduced or ameliorated include, but are not limited to, inflammation, gastrointestinal side effects (e.g., diarrhea, nausea, gastroenteritis, vomiting, gastrointestinal, rectal, and duodenal hemorrhage, hemorrhagic pancreatitis, large intestine perforation black or bloody stools, and/or coughing up blood); hematologic side effects (e.g., leucopenia, anemia, pancytopenia and agranulocytosis, thrombocytopenia, neutropenia, pure red cell aplasia (PRCA), deep venous thrombosis easy bruising, and/or unusual bleeding from the nose, mouth, vagina, or rectum); immunologic side effects/clinical manifestations (e.g., immunosuppression, immunosuppression resulting in sepsis, opportunistic infections (herpes simplex virus, herpes zoster, and invasive candidal infections), and/or increased infection); oncologic side effects/clinical manifestations (e.g., lymphoma, lymphoproliferative disease and/or non-melanoma skin carcinoma); renal side effects/clinical manifestations (e.g. dysuria, urgency, urinary tract infections, hematuria, kidney tubular necrosis, and/or BK virus-associated nephropathy); metabolic side effects/clinical manifestations (e.g. edema, hyperphosphatemia, hypokalemia, hyperglycemia, hyperkalemia. swelling, rapid weight gain, and/or enlarged thyroid); respiratory side effects/clinical manifestations (e.g., respiratory infection, dyspnea, increased cough, primary tuberculosis dry cough, wheezing, and/or stuffy nose); dermatologic side effects/clinical manifestations (e.g., acne, rash, dyshidrotic eczema, papulosquamous psoriatic-like skin eruption rash, blisters, oozing, mouth sores, and/or hair loss); muscoskeletal side effects/clinical manifestations (e.g. myopathy and/or muscle pain), hepatic side effects/clinical manifestations (e.g. hepatoxicity and/or jaundice), abdominal pain, increased incidence of first trimester pregnancy loss, missed menstrual periods, severe headache, confusion, change in mental status, vision loss, seizure (convulsions), increased sensitivity to light, dry eye, red eye, itchy eye, and/or high blood pressure. As provided above, the reduction or amelioration of the side effect or clinical manifestation is a reduction or amelioration, as compared to the severity of the side effect or clinical manifestation prior to administration of the drug formulation to the SCS of the eye of the patient, or a reduction or amelioration of the side effect or clinical manifestation in the patient, as compared to the reduction or amelioration experienced upon intravitreal, intracameral, parenteral or oral administration of the same drug.

The term “resolution” as used herein refers to a return to a baseline level. For example, in some embodiments, resolution of macular thickening or resolution of macular edema and the like is defined as a macular thickness of less than 300 microns in CST. In some embodiments the resolution of inflammation refers to a score of zero with respect to inflammatory measures in the eye. For example, in some embodiments, resolution of inflammation in the eye refers to a score of 0 of anterior chamber flare and/or anterior chamber cells and/or vitreous haze and/or other measures of eye inflammation known in the art.

The “therapeutic formulation” delivered via the methods and devices provided herein in one embodiment, is an aqueous solution or suspension, and comprises an effective amount of the drug or therapeutic agent, for example, a cellular suspension. In some embodiments, the therapeutic formulation is a drug formulation. The “drug formulation” is a formulation of a drug, which typically includes one or more pharmaceutically acceptable excipient materials known in the art. The term “excipient” refers to any non-active ingredient of the formulation intended to facilitate handling, stability, dispersibility, wettability, release kinetics, and/or injection of the drug. In one embodiment, the excipient may include or consist of water or saline.

The therapeutic formulation delivered to the suprachoroidal space of the eye of a human subject for the treatment of macular edema associated with uveitis (e.g., non-infectious uveitis of any anatomical subtype), may be in the form of a liquid drug, a liquid solution that includes a drug or therapy in a suitable solvent, or liquid suspension. The liquid suspension may include microparticles or nanoparticles dispersed in a suitable liquid vehicle for infusion. In various embodiments, the drug is included in a liquid vehicle, in microparticles or nanoparticles, or in both the vehicle and particles. The drug formulation is sufficiently fluid to flow into and within the suprachoroidal space, as well as into the surrounding posterior ocular tissues. In one embodiment, the viscosity of the drug formulation is about 1 cP at 37° C. In some embodiments, the viscosity of the drug formulation is from about 2 to about 40 cPs at 25° C. In further embodiments, the viscosity of the drug formulation is from about 5 to about 20 cPs at 25° C. In further embodiments, the viscosity of the drug formulation is from about 6 to about 15 cPs at 25° C.

In one embodiment, the drug formulation is a fluid and/or suspension. In one embodiment, the drug formulation includes microparticles or nanoparticles, either of which includes at least one drug. Desirably, the microparticles or nanoparticles provide for the controlled release of drug into the suprachoroidal space and surrounding posterior ocular tissue. In some embodiments, the drug formulation is prepared with starting material comprising microparticles or nanoparticles, either of which includes at least one drug. As used herein, the term “microparticle” encompasses microspheres, microcapsules, microparticles, and beads, having a number average diameter of from about 1 μm to about 200 μm, for example from about 1 to about 100 μm, or from about 1 μm to about 25 μm or from about 1 μm to about 7 μm. “Nanoparticles” are particles having an average diameter of from about 1 nm to about 1000 nm. The microparticles, in one embodiment, have a D₅₀ of about 5 μm or less. In a further embodiment the microparticles have a D₅₀ of less than 5 μm. In a further embodiment, the D₅₀ is about 3 μm or less. In a further embodiment, the D₅₀ is about 2 μm. The microparticles, in one embodiment, have a D₅₀ of about 1 μm to about 5 μm. In another embodiment, the D₅₀ of the particles in the drug formulation is about 2 μm or less. In another embodiment, the D₅₀ of the particles in the drug formulation is about 1000 nm or less. In another embodiment, the D₅₀ of the particles in the drug formulation is about 100 nm to about 1000 nm. In one embodiment, the drug formulation comprises microparticles having a D₉₉ of about 10 μm or less. In one embodiment, the drug formulation comprises microparticles having a D₉₉ of about 1000 nm to about 10 μm. In another embodiment, the D₉₉ of the particles in the formulation is less than about 10 μm, or less than about 9 μm, or less than about 7 μm or less than about 3 μm. In a further embodiment, the microparticles or nanoparticles comprise an anti-inflammatory drug. In a further embodiment, the anti-inflammatory drug is triamcinolone.

Microparticles and nanoparticles may or may not be spherical in shape. “Microcapsules” and “nanocapsules” are defined as microparticles and nanoparticles having an outer shell surrounding a core of another material. The core can be liquid, gel, solid, gas, or a combination thereof. In one case, the microcapsule or nanocapsule may be a “microbubble” or “nanobubble” having an outer shell surrounding a core of gas, wherein the drug is disposed on the surface of the outer shell, in the outer shell itself, or in the core. (Microbubbles and nanobubbles may be respond to vibrations as known in the art for diagnosis or to burst the microbubble to release its payload at/into a select ocular tissue site.) “Microspheres” and “nanospheres” can be solid spheres, can be porous and include a sponge-like or honeycomb structure formed by pores or voids in a matrix material or shell, or can include multiple discrete voids in a matrix material or shell. The microparticles or nanoparticles may further include a matrix material. The shell or matrix material may be a polymer, amino acid, saccharide, or other material known in the art of microencapsulation.

The drug-containing microparticles or nanoparticles may be suspended in an aqueous or non-aqueous liquid vehicle. The liquid vehicle may be a pharmaceutically acceptable aqueous solution, and optionally may further include a surfactant. The microparticles or nanoparticles of drug themselves may include an excipient material, such as a polymer, a polysaccharide, a surfactant, etc., which are known in the art to control the kinetics of drug release from particles.

The therapeutic substance in one embodiment is formulated with one or more polymeric excipients to limit therapeutic substance migration and/or to increase viscosity of the formulation. A polymeric excipient may be selected and formulated to act as a viscous gel-like material in-situ and thereby spread into a region of the suprachoroidal space and uniformly distribute and retain the drug. The polymer excipient in one embodiment is selected and formulated to provide the appropriate viscosity, flow and dissolution properties. For example, carboxymethylcellulose is used in one embodiment to form a gel-like material in the suprachoroidal space. The viscosity of the polymer in one embodiment is enhanced by appropriate chemical modification to the polymer to increase associative properties such as the addition of hydrophobic moieties, the selection of higher molecular weight polymer or by formulation with appropriate surfactants.

The dissolution properties of the therapeutic formulation in one embodiment is adjusted by tailoring of the water solubility, molecular weight, and concentration of the polymeric excipient in the range of appropriate thixotropic properties to allow both delivery through a small gauge needle and localization in the suprachoroidal space. The polymeric excipient may be formulated to increase in viscosity or to cross-link after delivery to further limit migration or dissolution of the material and incorporated drug.

Water soluble polymers that are physiologically compatible are suitable for use as polymeric excipients in the therapeutic formulations described herein, and for delivery via the methods and devices described herein include but are not limited to synthetic polymers such as polyvinylalcohol, polyvinylpyrollidone, polyethylene glycol, polyethylene oxide, polyhydroxyethylmethacrylate, polypropylene glycol and propylene oxide, and biological polymers such as cellulose derivatives, chitin derivatives, alginate, gelatin, starch derivatives, hyaluronic acid, chondroiten sulfate, dermatin sulfate, and other glycosoaminoglycans, and mixtures or copolymers of such polymers. The polymeric excipient is selected in one embodiment to allow dissolution over time, with the rate controlled by the concentration, molecular weight, water solubility, crosslinking, enzyme lability and tissue adhesive properties of the polymer.

In one embodiment, a viscosity modifying agent is present in a therapeutic formulation delivered by one of the methods and/or devices described herein. In a further embodiment, the viscosity modifying agent is polyvinyl alcohol, polyvinyl pyrrolidone, methyl cellulose, hydroxypropyl cellulose, hydroxyethyl cellulose, hydroxymethyl cellulose or hydroxypropyl cellulose. In another embodiment, the formulation comprises a gelling agent such as poly(hydroxymethylmethacrylate), poly(N-vinylpyrrolidone), polyvinyl alcohol or an acrylic acid polymer such as Carbopol.

As described above, the drug formulation delivered to the suprachoroidal space via the methods described herein can be administered with one or more additional drugs. The one or more additional drugs, in one embodiment, are present in the same formulation as the initial drug formulation. In another embodiment, the one or more additional drugs are present in a second formulation. In even a further embodiment, the second drug formulation is delivered to the patient in need thereof via a non-surgical SCS delivery method described herein. Alternatively, the second drug formulation is delivered intravitreally, intracamerally, sub-tenonally, orally, topically or parenterally to the human subject. In one embodiment, a VEGF antagonist is delivered to the suprachoroidal space of the eye of a human subject via one of the methods and/or devices disclosed herein, in conjunction with an anti-inflammatory compound.

As described above, in addition to suprachoroidal delivery, the one or more additional drugs delivered to the human subject can be delivered via intravitreal (IVT) administration (e.g., intravitreal injection, intravitreal implant or eye drops). Methods of IVT administration are well known in the art. Examples of classes of drugs that can be administered via IVT include, but are not limited to: VEGF modulators, PDGF modulators, anti-inflammatory drugs. Accordingly, the methods of the present invention include administrating via IVT one or more of the drugs listed above in combination with one or more drugs disclosed herein administered into the suprachoroidal space using the microneedle device described herein.

### EXAMPLES

The present invention is further illustrated by reference to the following Examples. However, it should be noted that these Examples, like the embodiments described above, are illustrative and are not to be construed as restricting the scope of the invention in any way.

### Example 1. Triamcinolone Formulations for Delivery to the Suprachoroidal Space

Triamcinolone is delivered to the suprachoroidal space using the methods and devices provided herein. The triamcinolone formulation, in one embodiment, is selected from one of the following seven formulations in Table 2. In one embodiment, the formulation is delivered in a volume of 100 μL for total administrated dose of 4 mg TA per administration.

TABLE 2
Table 2. Triamcinolone (TA) formulations.

| | Formulation | Formulation | Formulation | Formulation |
|---|---|---|---|---|
| Ingredient | A | B | C | D |
| Triamcinolone acetonide | 40 mg/mL | 40 mg/mL | 40 mg/mL | 40 mg/mL |
| Particle Size | D₅₀: ~2 μm | D₅₀: ~2 μm | D₅₀: ~2 μm | D₅₀: ~2 μm |
| | D₉₉: <10 μm | D₉₉: <10 μm | D₉₉: <10 μm | D₉₉: <10 μm |
| Sodium Chloride | 0.64% w/v | 0.64% w/v | 0.64% w/v | 0.55% w/v |
| Carboxymethylcellulose | 0.5% w/v | 0.5% w/v | 0.5% w/v | 0.5% w/v |
| sodium | | | | |
| Polysorbate 80 | 0.02% w/v | 0.015% w/v | ≥0.015% w/v | 0.015% w/v |
| KCl | 0.075% w/v | 0.075% w/v | 0.075% w/v | 0.075% w/v |
| CaCl₂ (dihydrate) | 0.048% w/v | 0.048% w/v | 0.048% w/v | 0.048% w/v |
| MgCl₂ (hexahydrate) | 0.030% w/v | 0.030% w/v | 0.030% w/v | 0.030% w/v |
| Sodium acetate | 0.39% w/v | 0.39% w/v | 0.39% w/v | 0.39% w/v |
| (trihydrate) | | | | |
| Sodium citrate (dihydrate) | 0.17% w/v | 0.17% w/v | 0.17% w/v | 0.17% w/v |
| NaOH/HCl | Adjust to pH | Adjust to pH | Adjust to pH | Adjust to pH |
| | 6.0-7.5 | 6.0-7.5 | 6.0-7.5 | 6.0-7.5 |

| | Formulation | Formulation | Formulation |
|---|---|---|---|
| Ingredient | E | F | G |
| Triamcinolone acetonide | 40 mg/mL | 40 mg/mL | 8 mg/mL |
| Particle Size | D₅₀: ~2 μm | D₅₀: ~2 μm | D₅₀: ~2 μm |
| | D₉₉: <10 μm | D₉₉: <10 μm | D₉₉: <10 μm |
| Sodium Chloride | 0.64% w/v | 0.55% w/v | 0.55% w/v |
| Carboxymethylcellulose | 0.5% w/v | 0.5% w/v | 0.5% w/v |
| sodium | | | |
| Polysorbate 80 | 0.02% w/v | 0.02% w/v | 0.01% w/v |
| KCl | 0.075% w/v | 0.075% w/v | 0.075% w/v |
| CaCl₂ (dihydrate) | 0.048% w/v | 0.048% w/v | 0.048% w/v |
| MgCl₂ (hexahydrate) | 0.030% w/v | 0.030% w/v | 0.030% w/v |
| Sodium acetate | 0.39% w/v | 0.39% w/v | 0.39% w/v |
| (trihydrate) | | | |
| Sodium citrate (dihydrate) | 0.17% w/v | 0.17% w/v | 0.17% w/v |
| NaOH/HCl | Adjust to pH | Adjust to pH | Adjust to pH |
| | 6.0-7.5 | 6.0-7.5 | 6.0-7.5 |

### Example 2. Clinical Evaluation of SCS-TA in Uveitis Patients

A two-arm, randomized, controlled, double-masked, multi-center Phase 3 clinical study was conducted to assess the effectiveness of two doses of SCS-TA in patients with non-infectious uveitis. Yeh S et al. Opthalmology 2020 127 (7): 948-955. The non-infectious uveitis included any anatomic subtype (anterior, intermediate, posterior, or panuveitis). The study is referred to as the PEACHTREE study in some embodiments herein. In the study, patients with non-infectious uveitis with macular edema due to their condition and reading≤70 ETDRS letters were randomly assigned to an active (SCS administration of SCS-TA) or control (sham SCS injection; no treatment) arm. The subject in the study could have uveitis with any disease diagnosis and etiology in the uveitis spectrum, and with any anatomic location for their uveitis: pan, intermediate, posterior, or anterior. Study medications were administered at day 0 (time 0) and at week 12, and subjects were evaluated every 4 weeks following the baseline treatment. In the study, 160 subjects with macular edema associated with noninfectious uveitis of any anatomic subtype (anterior, intermediate, posterior, or panuveitis) were randomized 3:2 either to suprachoroidal injections of SCS-TA (4.0 mg; n=96) or sham procedures (n=64).

The sham procedure was a sham SCS injection. In the SCS-TA treatment group, subjects were administered 4 mg SCS-TA by SCS injection (100 μL of a drug formulation comprising 40 mg/mL SCS-TA). The 4 mg SCS-TA treatment was administered as a single injection at 2 timepoints, one on day 0 and one on week 12. The sham procedure was also administered at day 0 and at week 12. Subjects were evaluated for a period of at least 6 months starting from day 0. Inclusion and exclusion criteria are included the following.

### Inclusion:

Diagnosis of non-infectious uveitis with any etiology and disease diagnosis within the uveitis spectrum of diseases
Any geographic location of uveitis: pan, anterior, intermediate, and posterior
Diagnosis of macular edema associated with non-infectious uveitis
Visual acuity score of ≥5 letters read (20/800 Snellen equivalent) and ≤70 letters read (20/40 Snellen equivalent), in the study eye

### Exclusion:

Any active ocular disease or infection in the study eye other than uveitis
Intraocular pressure >22 mmHg or uncontrolled glaucoma in the study eye for patients in the US and Israel; no increased IOP or glaucoma for subjects in India
Any uncontrolled systemic disease that, in the opinion of the Investigator, would preclude participation in the study

The primary endpoint of the study was the proportion of patients in each arm (SCS-TA treatment group vs. sham procedure control) gaining ≥15 ETDRS letters in BCVA from baseline at week 24. Strikingly, 46.9% of subjects in the treatment arm gained at least 15 ETDRS letters in BCVA by week 24. In contrast, only 15.6% of the sham control patients reached at least 15 ETDRS letters in BCVA by week 24. The difference between the two groups was highly statistically significant.

The baseline ETDRS letters read was 54.7. In the SCS-TA arm and 53.6 in the sham control arm. The mean change in BCVA across the entire Active Arm group was an increase of 13.8 letters read, in contrast to just 3.0 letters read in the Control Arm group (p<0.001) The mean change in ETDRS letters read consistently increased over months 1, 2, 3, 4, 5, and 6 of the study, and a highly statistically significant difference (p<0.001) compared to the Control Arm at each time point. By week 4, subjects in the SCS-TA group (bars) already had a far larger increase from baseline in ETDRS letters read compared to the control group (lines). The difference between SCS-TA and control treated subjects remained significant at each of weeks 8, 12, 16, 20, and 24.

Central retinal thickness was also measured throughout the study. The baseline central retinal thickness (CRT) was 480.0 microns in the active arm, and 518.0 microns in the control arm. At week 24, the mean change in CST was a decrease in CST of 152.6 microns in the Active Arm, compared to just 17.9 microns in the Control Arm (p<0.001). Thus, the SCS-TA treatment provided a highly statistically significant difference in the central retinal thickness compared to control treated patients. Over 50% of the subjects in the Active Arm showed a 20% or greater reduction in excess CST. This was observed at week 4, and sustained through week 24. Moreover, approximately 50% or more of subjects in the Active Arm showed resolution of macular thickening (defined as less than 300 microns in CST). This too was observed at week 4 and sustained through week 24.

SCS-TA increased BCVA relative to the control arm in all four anatomic subtypes. SCS-TA also significantly reduced CST in all anatomic subtypes except for patients with anterior uveitis. Accordingly, the Phase 3 study showed that subjects having macular edema associated with noninfectious uveitis, SCS injection of SCS-TA improved patient outcomes irrespective of the anatomic location of uveitis.

The SCS-TA treatment also resulted in a resolution of inflammation. About 70-75% of patients in the Active Arm had scores of zero with respect to anterior chamber flare, cells present in the anterior chamber, and vitreous haze, each of which is a measure of inflammation. In contrast, only about 17-23% of patients in the control arm exhibited a resolution of inflammation.

Table 3 provides safety summary and safety details. All 160 patients in the study (96:64 active:control) were in the Safety population. There were 3 serious adverse events (SAEs); all three were considered unrelated to treatment. Elevated IOP occurred in 11.5% of subjects in the SCS-TA group and 15.6% of subjects in the control group. All IOP increases in the control group were associated with local corticosteroid rescue treatment. Elevated IOP was managed with topical IOP-lowering medication when treatment was necessary. Cataract AEs were comparable in subjects in the CLS TA arm (7.3%) and control arm (6.3%). In the SCS-TA arm, 13.5% of subjects required rescue medication relative to 71.9% for the control arm. Thus, SCS-TA is a safe and highly effective treatment for non-infectious uveitis.

TABLE 3
Treatment emergent adverse events (TEAEs)

| | SCS-TA | Sham | Total |
|---|---|---|---|
| TEAE | 107 | 64 | 171 |
| TEAE, related | 31 | 8 | 39 |

Together, the results showed that the primary endpoint was met, showing superiority in the proportion of patients in the active (SCS-TA) arm over the control (sham) arm in gaining 15 or more ETDRS letters in BCVA at week 24 from baseline, with P<0.001. Key secondary endpoints of mean change in central retinal thickness from baseline and mean change in BCVA were also met, with P<0.001. Approximately 1 in 2 (˜50%) of the uveitis patients who were administered SCS SCS-TA improved their vision from baseline by 15 or more ETDRS letters. Thus, the results of the Phase 3 clinical study indicated that SCS administration 4 mg of triamcinolone, administered in at least two dosing sessions spaced about 90 days apart, was highly effective, to a highly statistically significant degree compared to control treated patients, in improving vision and CST. In fact, most subjects treated with the claimed dosing regimen experienced a resolution of macular thickening. In addition, the method was highly effective in resolving inflammation of the eye.

In summary, in this pivotal, 6-month, phase 3 trial in subjects with uveitic macular edema of any anatomic location, suprachoroidal injection of SCS-TA met its primary endpoint (% of subjects gaining ≥15 ETDRS letters in BCVA) when compared to sham control while also demonstrating a favorable safety profile.

A further study was conducted to assess the durability of the effect of SCS-TA administration to the SCS. The design of the study was a prospective, non-interventional, masked, observational 24-week extension trial from the 24 week study described above. To be eligible, subjects completed the study described above and did not receive rescue medication. The exit visit for the initial 24-week study was the first visit for the extension trial. Of the 61 (40%) potential subjects from the initial 24 week study at the sites where the extension study was offered, a total of 33 subjects were enrolled. 28 were in the SCS-TA arm and 5 were in the control arm. Subjects were evaluated every 6 weeks for a maximum of 24 additional weeks; follow up from baseline of the initial 24 week study was therefore 48 weeks. The last dose of SCS-TA (or control) was at week 12 of the initial study. In the extension study, subjects were exited if they qualified to receive rescue medication or at week 48, whichever occurred first.

The objective of the extension trial was to assess the durability of SCS-TA in the uveitis subject population. A Kaplan-Meier evaluation was conducted. 28 SCS-TA patients and 5 Control patients were enrolled, and patient demographics, dispositions, and uveitis disease diagnoses were recorded. All patients were included in the safety population. Uveitis diagnoses included idiopathic, sarcoidosis, HLA-B27-related, juvenile idiopathic arthritis, birdshot retinochoroidopathy, pars plantis, and others. The primary outcome in the extension study was the time to rescue therapy relative to Day 0 of the initial 24 week study.

The efficacy results of the study showed that 50% of SCS-TA subjects did not receive any additional medication through week 48. Therefore, half of the enrolled SCS-TA subjects were able to go at least 36 weeks (9 months) from their last (second PEACHTREE protocol-mandated) dose of SCS-TA, indicating strong durability of the treatment. Mean time to rescue therapy was 344 days, from Day 0 of the initial 24 week study. Moreover, the subjects maintained improved vision and improved macular edema throughout. Subjects who entered the extension study at week 24 had gained 16-17 ETDRS letters from baseline; those who remained in the extension study through week 48 maintained gains of approximately 12 letters. Subjects who entered the extension study improved by approximately 170 microns in CST at week 24 from baseline; those who remained in the extension study through week 48 maintained these gains. Thus, the mean improvement in CST through week 48 was approximately 170 microns. Thus, the study showed that the SCS-TA treatment provided a significant, durable improvement in macular edema associated with uveitis.

Safety: There were no serious adverse events related to the study medication; elevations in IOP were consistent with those seen in the initial 24 week study and were low. There were only three elevated IOP adverse events of 10 mmHg or above from baseline in the initial 24 week study, and one in the extension study. No IOP adverse events were above 30 mmHg at any visit during the initial 24 week study, and only one during the extension study. Two elevated IOP adverse reactions were treated with IOP lowering drops, and no surgery was required for any of the IOP increases. There were 3 cataracts in the initial 24 week study and 4 more cataracts in the extension study, for a total of 7 in the active treatment group. There were 25 (7/28) and 20% (1/5) new cataract adverse events in the SCS-TA and control arms, respectively. For the cataract adverse events, there were two cataract surgeries performed.

Taken together, the results of the studies showed that SCS administration of SCS-TA provides a safe, effective, and durable treatment for macular edema subjects. Surprisingly, in those patients who did not require rescue treatment, the safety and improvement in vision and macular edema in patients administered SCS-TA lasted for at least 48 weeks from the initial dose of SCS-TA and at least 36 weeks from the last dose of SCS-TA, demonstrating a marked durability of the treatment.

### Example 3. Suprachoroidal Triamcinolone Acetonide Injectable Suspension for Macular Edema Associated with Uveitis: Effect of Disease Characteristics on Clinical Outcomes

Triamcinolone acetonide injectable suspension for suprachoroidal use (SCS-TA) provides targeted drug delivery to the choroid/retina while minimizing steroid exposure in nontarget tissues. Efficacy and safety of SCS-TA in the treatment of macular edema (ME) secondary to noninfectious uveitis (NIU) were demonstrated in PEACHTREE (described above in Example 2). The objective of these post-hoc analyses was to determine whether baseline disease characteristics impact the efficacy of SCS-TA. For example, in an embodiment, the effect of the time of treatment on clinical outcomes was assessed.

In PEACHTREE, 96 subjects received SCS-TA at baseline and Week 12; best-corrected visual acuity (BCVA) and central subfield thickness (CST; in μm) were evaluated every 4 weeks for 24 weeks. Mean [SEM] change from baseline (CFB) in BCVA (ETDRS letters) and CST (μm) were calculated among SCS-TA-treated subjects by time since ME diagnosis (n=24≤71 days, n=22>71 days) or NIU diagnosis (n=60≤177 days, n=36>177 days), and by disease onset (n=22 sudden, n=74 insidious) or duration (n=18≤3 months, n=78>3 months). Treatment differences were evaluated using an analysis of variance model with a fixed effect for baseline demographic factors.

Significant BCVA gains and CST reductions were found in all groups at all visits irrespective of time to diagnosis or disease onset duration (P≤0.05). BCVA gains over the study appeared greater in subjects with more recent diagnosis and earlier disease, with significant differences identified in the analysis. At Week 24, BCVA gains were greater in subjects with earlier diagnosis, with an increase of 15.5 [1.48] in subjects with ME diagnosis ≤71 days vs 8.0 [3.37] in those with diagnosis >71 days (P=0.024) and 19.6 [3.13] in subjects with NIU≤3 months vs 12.4 [1.55] with NIU>3 months (P=0.048). In subjects with NIU≤177 days vs >177 days and sudden vs insidious disease duration BCVA gains were 15.5 [1.70] vs 10.8 [2.42] and 18.4 [3.09] vs 12.4 [1.56], respectively at Week 24 (P≥0.075). In subjects with more recent vs earlier ME and NIU diagnoses, changes in CST were −164.2 [20.15] vs −115.3 [24.27] and −163.7 [23.28] vs −133.4 [20.15], while those with sudden vs insidious onset and duration ≤3 vs >3 months had CST values of −165.7 [38.50] vs −148.8 [18.25] and −166.3 [45.29] vs −149.3 [17.49], respectively (P≥0.210 for all).

The results of the study showed significant improvements in BCVA and CST regardless of baseline disease characteristics. Moreover, the results indicated trends towards greater improvements in those with more recent diagnosis. For example, greater gains in BCVA were associated with more recent ME diagnosis and shorter NIU duration. These findings suggest that treatment should be initiated promptly upon diagnosis.

### Example 4. Suprachoroidal Triamcinolone Acetonide Injectable Suspension for Macular Edema Associated with Uveitis: Visual and Anatomic Outcomes by Age

Safety and efficacy of triamcinolone acetonide injectable suspension, for suprachoroidal use (SCS-TA) in the treatment of macular edema (ME) associated with noninfectious uveitis (NIU) was previously demonstrated in the Phase 3 PEACHTREE study. This post-hoc analysis evaluated the impact of age on treatment outcomes from that study.

Subjects with ME secondary to NIU (N=160) were randomized 3:2 to receive a suprachoroidal injection of SCS-TA (4 mg) or a sham procedure in the study eye at baseline and Week 12 and were followed for 24 weeks. Subjects received rescue therapy based on preestablished criteria. Anatomical and visual outcomes, incidence of adverse events, and intraocular pressure (IOP) elevations were evaluated at each visit. A post-hoc analysis was performed to stratify best corrected visual acuity (BCVA, EDTRS letters), central subfield thickness (CST, μm), need for rescue and incidences of IOP and cataract by subject age (≤50 and >50 years).

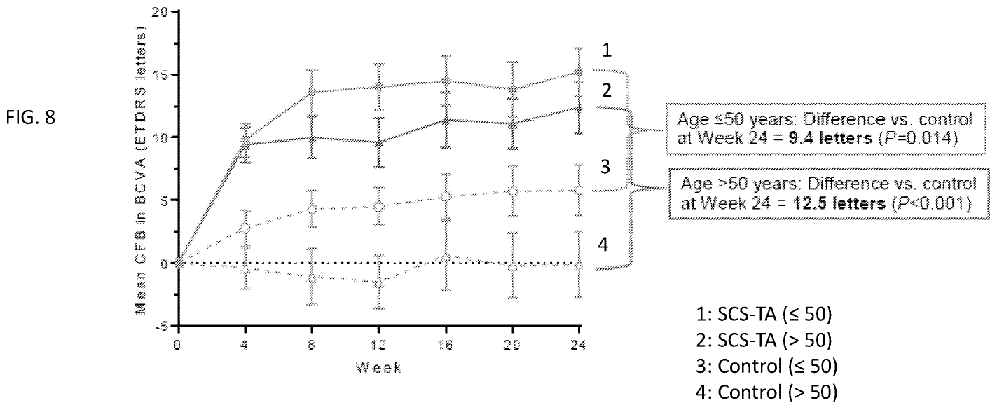

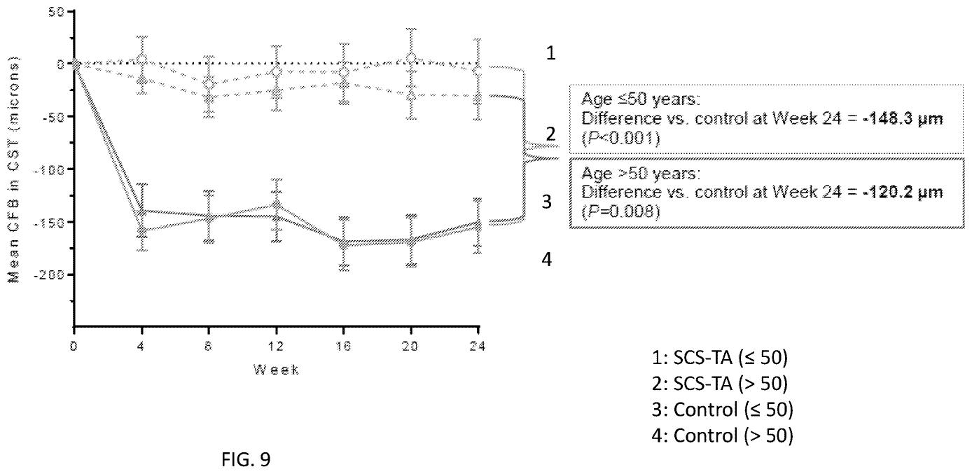

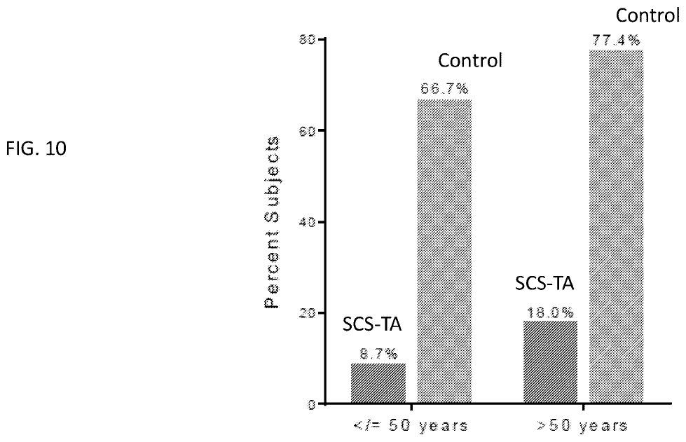

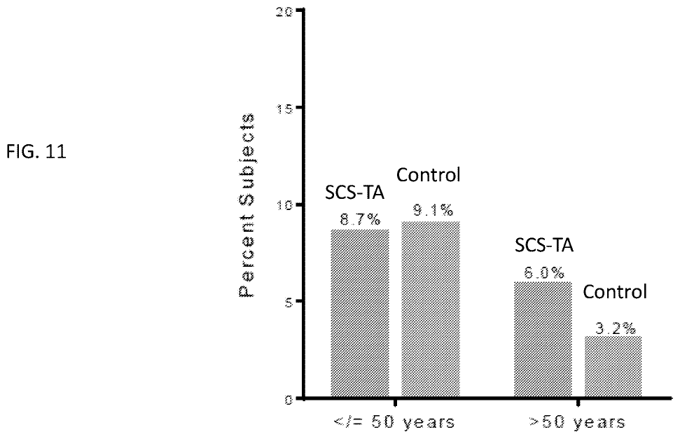

In the SCS-TA vs control arms, 46 vs 33 subjects were ≤50 years and 50 vs 31 subjects were >50 years of age. Similar proportions of subjects had persistent NIU (>3 month duration) in both age groups, while time since ME diagnosis was shorter in the younger age group (50.5 vs 90.0 weeks in the SCS-TA arm). Change from baseline (CFB) in BCVA was greater with SCS-TA vs control in both age groups at all visits. At Week 24, in subjects ≤50 and >50 years of age, respectively, the CFB in BCVA was 15.2 and 12.5 (P<0.001 for both) and between treatment differences were 9.4 and 12.5 (P<0.014 for both). The CFB in BCVA was highly statistically significant in the older age group (>50 years of age). In both age groups, CFB in CST was greater with SCS-TA vs control at all visits; between treatment differences were −148.3 and −120.2 in subjects ≤50 and >50 years of age (P≤0.008 for both). The rescue rate for SCS-TA vs control was 8.7 vs 66.7% and 18.0 vs 77.4% in subjects ≤50 and >50 years of age, respectively. Incidences of IOP elevation ≥10 mm Hg from baseline at any post-baseline visit in the SCS-TA vs control arms were 15.2 vs 18.2% and 16.3 vs 16.1% in the ≤50 and >50 year age groups, respectively. Cataract rates in the SCS-TA vs control arms were 8.7 vs 9.1% and 6.0 vs 3.2% in subjects ≤50 and >50 years of age, respectively. The data are further discussed in FIGS. 8-11. These data suggest that SCS-TA is effective and well tolerated in the treatment of ME associated with NIU irrespective of patient age.

### Example 5. Suprachoroidal Triamcinolone Acetonide Injectable Suspension for Macular Edema Associated with Uveitis: Outcomes by Anatomic Subtypes

Triamcinolone acetonide injectable suspension for suprachoroidal use (SCS-TA) provides targeted steroid delivery to the choroid/retina while minimizing exposure in nontarget tissues. The PEACHTREE Study evaluated SCS-TA for macular edema (ME) secondary to noninfectious uveitis (NIU). In these post-hoc analyses, we evaluated the efficacy and safety of SCS-TA in subjects from PEACHTREE categorized by discreet anatomic subtype of uveitis.

In PEACHTREE, 160 NIU subjects with ME were randomized 3:2 to SCS-TA or sham procedure at baseline and week 12 and were followed for 24 weeks. Subjects diagnosed with NIU of any anatomic subtype, including anterior-, intermediate-, posterior-, and panuveitis, were included. Herein, efficacy endpoints, including changes in best corrected visual acuity (BCVA), central subfield retinal thickness (CST), and safety endpoints, including adverse event (AE) reports and intraocular pressure (IOP), were evaluated in subjects diagnosed with NIU in a single anatomic location.

Of subjects in the SCS-TA and control arm, 13 vs 7 had anterior uveitis, 21 vs 15 had intermediate uveitis, 20 vs 11 had posterior uveitis, and 30 vs 24 had panuveitis. Across NIU subtypes, at week 24, SCS-TA-treated subjects showed significant improvements from baseline in BCVA ranging from 12.1 to 15.9 letters, while control subjects showed changes ranging from −1.6 to 9.1 letters, with significant between-treatment differences among subjects diagnosed with posterior or panuveitis (P≤0.024). Similarly, changes in CST ranging from −120.1 to −189.0 μm were observed in SCS-TA-treated subjects vs −20.3 to 10.2 μm in control subjects, with significant between-treatment differences among intermediate-, posterior- and panuveitis anatomic subtypes (P≤0.014). Reports of AEs appeared similar between treatments by anatomic subtype. At week 24, SCS-TA-treated subjects showed IOP changes of 0.5 to 3.1 mm Hg compared to −1.2 to 1.7 mm Hg in control subjects.

The results of the study indicated that treatment with SCS-TA resulted in significant BCVA improvements from baseline in all anatomic NIU subtypes and was significantly better vs control in posterior and panuveitis. Significant improvements in CST from baseline were observed vs both baseline and control for most anatomic NIU locations. Safety findings were comparable across discreet NIU subtypes.

### Example 6. Suprachoroidal Triamcinolone Acetonide Injectable Suspension for Macular Edema Associated with Uveitis: Integrated Analysis of Two Clinical Trials

Triamcinolone acetonide injectable suspension, for suprachoroidal use (SCS-TA) provides targeted drug delivery to the choroid and retina while minimizing steroid exposure in nontarget tissues. This integrated analysis evaluated SCS-TA for the treatment of macular edema (ME) secondary to noninfectious uveitis (NIU) across two studies.

Data from a randomized, double-masked, sham-controlled trial (PEACHTREE) and an open-label trial (AZALEA) were pooled. Only those subjects with ME secondary to NIU defined by a central subfield retinal thickness (CST)≥300 μm with fluid and a best-corrected visual acuity (BCVA) of ≥5 and ≤70 Early Treatment Diabetic Retinopathy Study (ETDRS) letters in the study eye at baseline were included in the analysis. In both studies, subjects received SCS-TA at baseline and Week 12 and were followed every 4 weeks for 24 weeks. Control subjects received a sham procedure at baseline and Week 12. Outcomes included BCVA, CST, anterior chamber (AC) cells, AC flare, vitreous haze (VH) and adverse events (AEs).

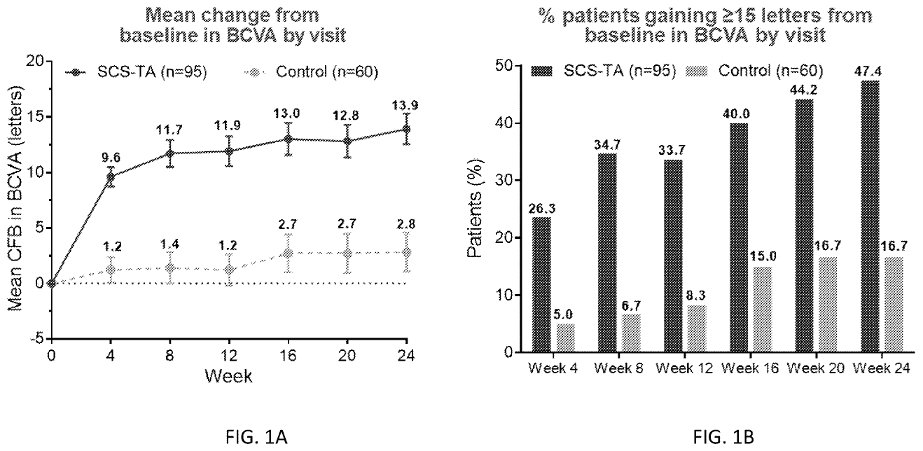

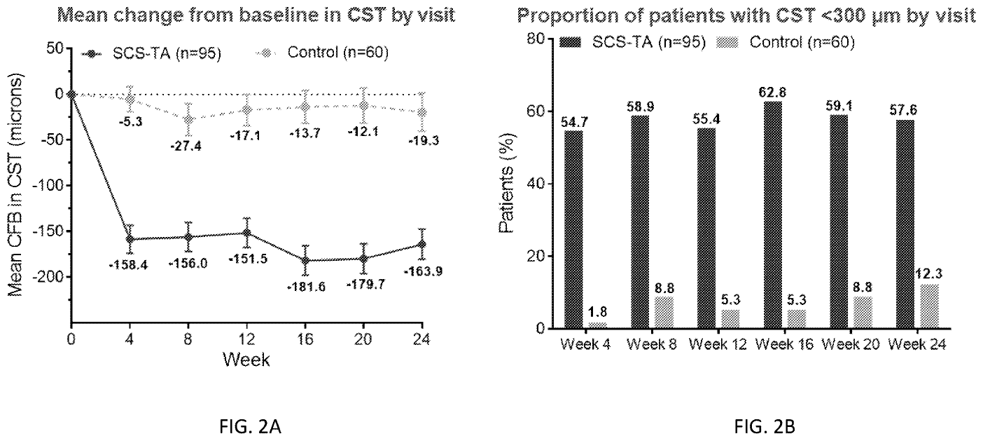

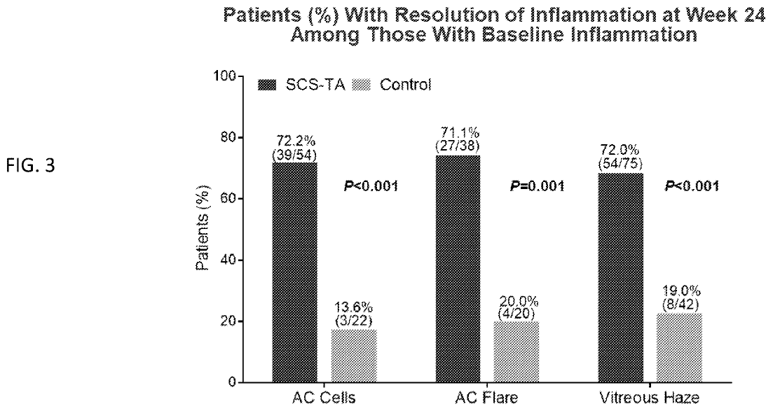

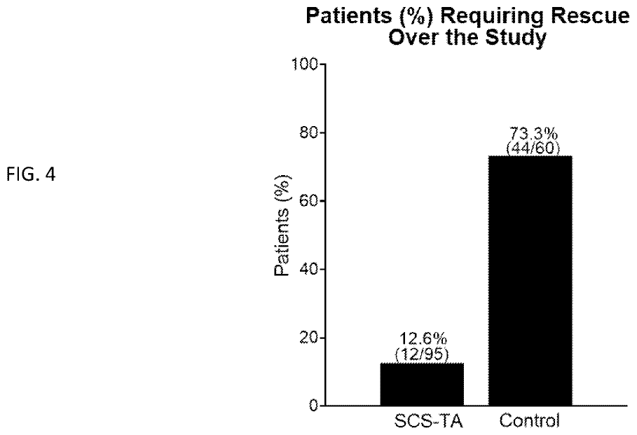

The integrated population included 95 SCS-TA subjects (PEACHTREE 88; AZALEA 7) and 60 control subjects. Increase from baseline in BCVA was greater with SCS-TA vs control with a mean difference of 10.7 letters at Week 24 (P<0.001 for all visits) (FIG. 1A). Proportions of subjects achieving a mean≥15 letters gain were greater with SCS-TA vs control (47.4% vs 16.7% at Week 24; P<0.001 for all visits), as shown in FIG. 1B. Reduction in CST was greater and more subjects achieved a CST<300 μm with SCS-TA vs control (P<0.001 for all visits), as shown in FIGS. 2A-B. In subjects with baseline inflammation, 72.2%, 71.1% and 72.0% in the SCS-TA group vs 13.6%, 20.0% and 19.0% in the control group achieved resolution of AC cells, flare and VH at Week 24 (all P<0.01) (FIG. 3). Rescue therapy was required by 12.6% and 73.3% of subjects in the SCS-TA and control groups, respectively (FIG. 4). AEs were mostly mild and no treatment-related serious AEs were reported. Overall, incidence of elevated intraocular pressure (IOP) was 20% and 15% in the SCS-TA and control groups (7.4% vs 0% at time of procedure; 12.6% vs 15% post-procedure, control IOP events occurred post rescue). Cataract rates were 7.4% and 6.7% for SCS-TA and control respectively. Significant improvement in vision represented by a mean visual acuity gain of 14 letters including a mean≥15 letters gain in almost 50% of SCS-TA subjects was noted as well as significant reductions in CST from baseline in those subjects. Reduction of AC cells, AC flare, and vitreous haze by Week 24 in the majority of patients who exhibited these signs at baseline was also confirmed.

In this integrated analysis of two Phase 3 clinical trials, SCS-TA was confirmed to be effective and well tolerated in the treatment of ME associated with NIU.

### Example 7. National Physician Survey on Clinical Practice Patterns for the Treatment of Noninfectious Uveitis

Noninfectious uveitis (NIU) often manifests as a persistent relapsing disease that may be challenging to treat. This survey was conducted to evaluate current treatment patterns for chronic and acute NIU among retina specialists and identify unmet needs associated with therapy for NIU and associated macular edema (ME).

An anonymized online survey on NIU practice patterns was designed and sent to ophthalmologists throughout the US. Screening questions limited respondents to retina specialists with retina/uveitis fellowship training and intravitreal injection experience. The survey consisted of questions related to respondent's location and specialty, occurrence of ME in association with NIU, NIU treatment patterns and therapeutic unmet needs. Responses were analyzed using descriptive statistics.

Survey respondents (N=150) were evenly distributed geographically. Respondents reported ME secondary to 31% and 61% of acute and persistent NIU cases respectively, with an overall rate of 47%. Corticosteroids were the most common initial treatment for acute NIU (>90% of cases), mostly administered topically. Proportions of patients with unilateral/bilateral acute NIU receiving topical corticosteroids were 93%/91%, 51%/47% and 47%/46% for anterior, posterior and pan NIU, respectively. In persistent NIU, therapy was typically changed to another corticosteroid and/or a non-steroidal immunosuppressant. Among patients receiving corticosteroids for persistent NIU, periorbital delivery was the most common route of administration for unilateral/bilateral anterior NIU (59%/55%), while intravitreal delivery was most common for posterior NIU (64%/59%) and pan NIU (68%/63%). Respondents refrained from using corticosteroids in 20% of patients due to concern over elevated intraocular pressure (IOP) and considered elevated IOP, glaucoma and cataract risk to be moderately high concerns when selecting therapy (ranking of 4.0, 3.9, 3.5, respectively, on a 1-5 scale). A lower risk of IOP elevation was ranked as the greatest unmet need for corticosteroid therapies.

Survey results indicate that ME is commonly associated with NIU, and local corticosteroids are typically used first-line. However, physicians may limit use of corticosteroids due to risk of associated side effects such as cataract and IOP elevation; effective therapies with lower incidence of these adverse reactions are warranted.

### Example 8. Efficacy of Suprachoroidal Triamcinolone Acetonide Injectable Suspension in the Treatment of Macular Edema in Patients with Chronic Uveitis

This post-hoc analysis evaluated outcomes following treatment with triamcinolone acetonide injectable suspension, for suprachoroidal use in patients with macular edema associated with chronic uveitis.

Safety and efficacy of triamcinolone acetonide injectable suspension, for suprachoroidal use (SCS-TA) in the treatment of macular edema (ME) associated with noninfectious uveitis (NIU) was previously demonstrated in PEACHTREE. This post-hoc analysis of PEACHTREE data evaluated outcomes in a subset of patients with chronic uveitis (persistent uveitis with relapse in <3 months after discontinuing prior treatment).

In PEACHTREE, subjects with ME secondary to NIU (N=160) were randomized 3:2 to receive a suprachoroidal injection of SCS-TA (4 mg) or a sham procedure in the study eye at baseline and Week 12. Anatomical, visual and safety outcomes were evaluated over 24 weeks. In this posthoc analysis, BCVA (EDTRS letters) and CST (μm) were assessed in SCS-TA subjects characterized as having chronic uveitis at baseline. Time to rescue in these subjects compared to control sham treated subjects was also evaluated. Changes from baseline for BCVA and CST at each visit were analyzed by t-test while any between treatment differences in time to rescue were analyzed using Kaplan-Meier estimates (Log-rank test).

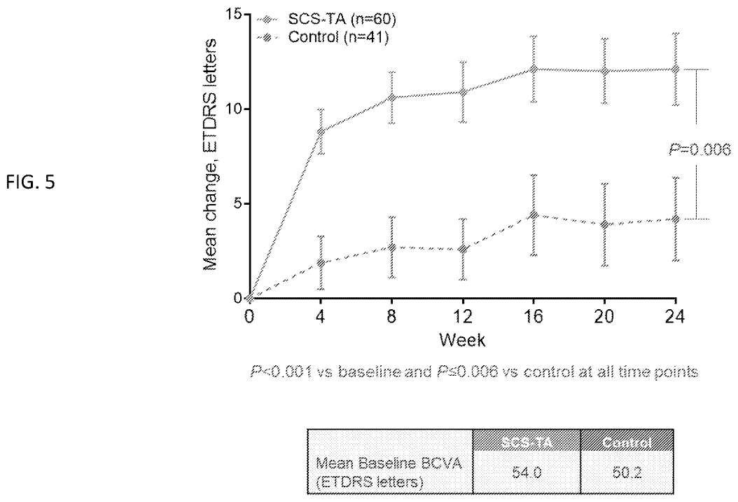

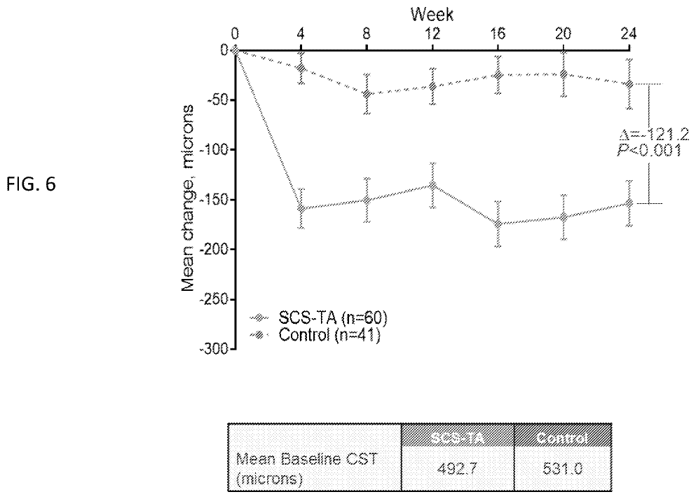

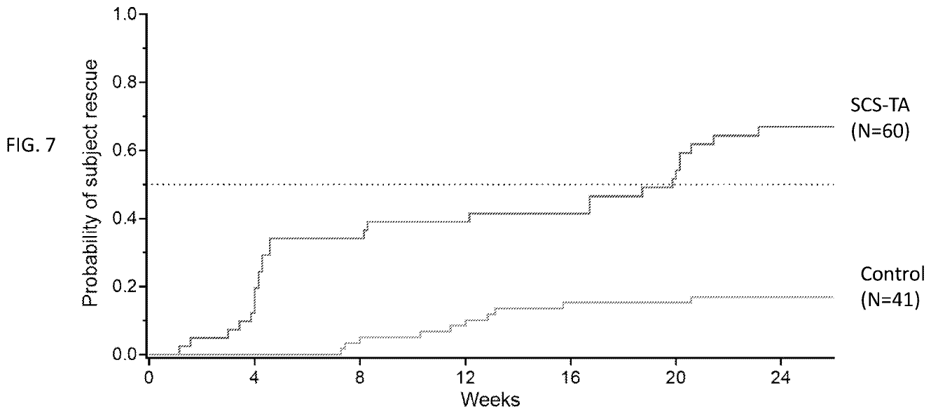

At baseline, 60 subjects treated with SCS-TA were characterized as having chronic uveitis. Mean age of SCS-TA subjects in this cohort was 50 years, the majority were female, and mean time since diagnosis was 188 days. Mean (SE) BCVA letters and CST (μm) at baseline were 54.0 (1.86) and 492.7 (19.88), respectively. Mean (SE) BCVA letter gains were 8.8 (1.18), 10.6 (1.35), 10.9 (1.59), 12.1 (1.72), 12.0 (1.69) and 12.1 (1.89) at Week 4, 8, 12, 16, 20, and 24, respectively (P<0.001 vs baseline at all visits). In comparison, mean (SE) BCVA letters and CST (μm) at baseline were 50.2 and 531.0, respectively. As shown in FIG. 5, increase in BCVA from baseline was greater with SCS-TA vs control showing a mean difference of 8.0 letters at Week 24. Similarly, mean (SE) reductions in CST (μm) of −158.7 (19.39), −150.5 (21.67), −135.4 (22.32), −174.3 (22.55), −167.4 (22.18, and −153.5 (22.58) were observed at these visits (P<0.001 vs baseline at all visits), showing a mean difference of −121.2 between SCS-TA vs control at Week 24 (FIG. 6). Of SCS-TA treated subjects with chronic uveitis, 16.7% (10/60) required recue compared to 65.9% (27/41) of control subjects with chronic uveitis (FIG. 7). Mean (SE) time to rescue among subjects requiring rescue was 133.7 (3.49) and 102.4 (9.67) in SCS-TA treated and control subjects, respectively (P<0.001).

Consistent with the full dataset, in this post-hoc analysis of pivotal Phase 3 data specific to subjects with chronic uveitis at baseline, SCS-TA was similarly effective in improving visual and anatomical outcomes in the treatment of ME associated with NIU as in the full data set. Few subjects required rescue.

### Example 9. Suprachoroidal Triamcinolone Acetonide Injectable Suspension: Impact on Uveitis Inflammation Indicators Beyond Macular Edema

This study reports outcomes for uveitis inflammation indicators following treatment with triamcinolone acetonide injectable suspension, for suprachoroidal use in subjects with macular edema associated with uveitis.

Safety and efficacy of triamcinolone acetonide injectable suspension, for suprachoroidal use (SCS-TA) in the treatment of macular edema (ME) associated with noninfectious uveitis (NIU) was demonstrated in the Phase 3 PEACHTREE study. Here we report on additional outcomes for uveitis signs of vitreous haze, anterior chamber (AC) cells, and AC flare among subjects enrolled in PEACHTREE.

In PEACHTREE, subjects with ME secondary to NIU (N=160) were randomized 3:2 to receive a suprachoroidal injection of SCS-TA (4 mg) or a sham injection procedure in the study eye at baseline and Week 12 and were followed for 24 weeks. At each visit, anterior chamber (AC) cells and AC flare were assessed via slit lamp, while vitreous haze was assessed using indirect ophthalmoscopy. All were graded on a scale of 0 to 4+, with AC cells and flare grading based on Standardization of Uveitis Nomenclature (SUN) criteria and vitreous haze grading based on standardized photographs. In this post hoc analysis, proportions of subjects with resolution of inflammation (score of 0) at Week 24 and proportions of subjects with a reduction in inflammation of ≥2 steps at Week 24 were determined. Differences between treatments were analyzed using Cochran-Mantel-Haenszel tests.

At baseline 25.6%, 35.6%, 21.9% and 32.5% of subjects were classified as having anterior-, intermediate-, posterior-, and pan-uvietis, with similar proportions in the SCS-TA treatment and control (i.e., sham procedure) arm. As well, at baseline, AC cells, AC flare and vitreous haze was reported for 52.1%/35.9%, 36.5%/31.2%, and 79.2%/68.7% of subjects in the SCS-TA arm and control arm, respectively. Of subjects with any level of AC cells at baseline, AC flare at baseline, or vitreous haze at baseline, respectively, 72.0%, 74.3% and 68.4. % of SCS-TA subjects had their inflammation resolved by Week 24 compared to 17.4%, 20.0%, and 22.7% of control subjects (P<0.001 for all). Similar results were noted at earlier study visits and when analyzing proportions with ≥2 steps decreases in inflammation.

The study indicated that a majority of subjects in PEACHTREE receiving SCS-TA with active inflammation at baseline experienced resolution of inflammation. SCS-TA may be useful in subjects with ME associated with NIU who also experience active uveitic inflammation.

### Example 10. Suprachoroidal Use of Triamcinolone Acetonide: Evaluation of Elevations in Intraocular Pressure Overall and Longitudinally

Triamcinolone acetonide injectable suspension, for suprachoroidal use (SCS-TA) provides targeted drug delivery to the choroid/retina while minimizing steroid exposure in nontarget tissues. Safety and efficacy of SCS-TA in the treatment of macular edema (ME) associated with noninfectious uveitis (NIU) was demonstrated in PEACHTREE. Here we evaluated elevations in intraocular pressure (IOP) among SCS-TA subjects and control subjects overall and longitudinally, by visit.

In PEACHTREE, subjects with ME associate with NIU (N=160) were randomized 3:2 to receive a suprachoroidal injection of SCS-TA (4 mg) or a sham procedure at baseline and Week 12 and followed for 24 weeks. Subjects could receive rescue therapy based on predetermined criteria. In this post hoc analysis, IOP elevations ≥10 mm Hg (excluding those values measured immediately following the injection procedure) were determined for subjects categorized by treatment and receipt of rescue. Proportions of subjects requiring ≥1 additional IOP-lowering medication or IOP-lowering surgery were summarized.

In PEACHTREE, 83 SCS-TA subjects and 18 control subjects completed the study without rescue (unrescued SCS-TA and unrescued control); 46 control subjects required rescue (rescued control). First rescue in the control group consisted of corticosteroids administered topically (39.1%), intravitreally (30.4%), systemically (13.0%), or periocularly (10.9%). Overall, IVT or periocular corticosteroids were used in 80.4% of rescued control subjects. At week 24, unrescued SCS-TA subjects showed a mean (SE) change from baseline of +1.6 (0.45) mm Hg compared with +2.0 (0.73) mm Hg in rescued control subjects and −1.2 (0.88) mm Hg in the unrescued control subjects. Over the study, unrescued SCS-TA subjects experienced fewer IOP elevations ≥10 mm Hg from baseline than control subjects who ever received rescued (12.2% vs. 23.9%, respectively), whereas none of the unrescued control subjects experienced an IOP elevation. Longitudinal analysis showed the proportion of subjects with IOP elevations was ≤9.5% in unrescued SCS-TA subjects at any visit compared with ≤3.6% in control subjects prior to rescue and ≤13.3% in rescued control subjects. IOP elevations in the rescued control group were temporally linked with the use of rescue medication. IOP-lowering medication was reported for 7.2% of unrescued SCS-TA vs 13.0% of rescued control subjects and none of the unrescued control subjects. No subjects underwent IOP-lowering surgery.

SCS-TA subjects showed a lower incidence of IOP elevations compared to rescued control subjects, despite the relatively shorter duration of treatment with rescue in control subjects. Direct head-to-head studies may determine true differences in IOP effects between SCS-TA and other local corticosteroid treatment options used in uveitic ME.

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

1. A method of treating macular edema associated with uveitis in a subject in need thereof, the method comprising non-surgically administering an effective amount of a triamcinolone drug formulation to the suprachoroidal space (SCS) of the eye of the human subject in need of treatment, wherein the uveitis is noninfectious uveitis of posterior, intermediate, or panuveitis anatomic subtype.

2. The method of claim 1, wherein the effective amount of triamcinolone in the drug formulation is about 2 mg to about 5 mg.

3. The method of claim 1, wherein the effective amount of triamcinolone in the drug formulation is about 4 mg.

4. The method of claim 1, wherein the triamcinolone is triamcinolone acetonide.

5. The method of claim 4, wherein the triamcinolone acetonide is formulated at 40 mg/mL.

6. The method of claim 4 or 5, wherein the triamcinolone acetonide is formulated as a suspension of microparticles having a D₅₀ of 3 μm or less.

7. The method of any one of claims 1-6, wherein the formulation comprises 0.02% (w/v) polysorbate 80 and 0.5% (w/v) carboxymethylcellulose sodium.

8. The method of any one of claims 1-7, wherein the method comprises administering two doses of the triamcinolone drug formulation to the SCS of the eye of the subject.

9. The method of claim 8, wherein the two doses of the triamcinolone drug formulation are administered about 1, 2, 3, 4, 5, 6, 7, 8, 9, 10, 11, 12, 13, 14, 15, 16, or 17 weeks apart.

10. The method of claim 8 or 9, wherein the two doses of the triamcinolone drug formulation are administered about 12 weeks apart.

11. The method of claim 8, wherein the two doses of the triamcinolone drug formulation are administered about 90 days apart.

12. The method of any one of claims 1-11, wherein the uveitis is posterior uveitis.

13. The method of any one of claims 1-11, wherein the uveitis is intermediate uveitis.

14. The method of any one of claims 1-11, wherein the uveitis is panuveitis.

15. The method of any one of claims 1-14, wherein the subject has a visual acuity score of between about 5 and about 70 letters read before administration of the triamcinolone drug formulation.

16. The method of any one of claims 1-15, wherein the method decreases retina thickness and/or macula thickness relative to a baseline measurement prior to treatment of the subject with the triamcinolone drug formulation.

17. The method of any one of claims 1-16, wherein the method decreases retina thickness and/or macula thickness relative to a subject that did not receive the triamcinolone drug formulation.

18. The method of claim 16 or 17, wherein the retinal thickness is decreased by at least about 20 μm, at least about 40 μm, at least about 50 μm, at least about 100 μm, at least about 125 μm, least about 150 μm, at least about 175 μm, or at least about 200 μm.

19. The method of any one of claims 16-18, wherein the decrease in retinal thickness and/or macula thickness is maintained for at least 36 weeks following the last dose of triamcinolone drug formulation to the SCS.

20. The method of any one of claims 16-18, wherein the decrease in retinal thickness and/or macula thickness is maintained at for at least 48 weeks following the first dose of triamcinolone drug formulation to the SCS.

21. The method of any one of claims 1-20, wherein the method results in resolution of macular edema by about week 4 following administration of the first dose of triamcinolone drug formulation to the SCS.

22. The method of claim 21, wherein the resolution of macular edema is maintained for at least 24 weeks following administration of the first dose of triamcinolone drug formulation to the SCS.

23. The method of any one of claims 1-22, wherein the method results in resolution of inflammation in the eye.

24. The method of claim 23, wherein the resolution of inflammation comprises a reduction of 2 or more scores in anterior chamber flare, cells present in the anterior chamber, and/or vitreous haze at 24 week following administration of the first dose of triamcinolone drug formulation.

25. The method of claim 23, wherein the resolution of inflammation comprises a reduction of eye inflammation score to zero.

26. The method of any one of claims 1-25, wherein the method increases a Best Corrected Visual Acuity (BCVA) of the subject relative to a baseline measurement prior to treatment of the subject with triamcinolone drug formulation.

27. The method of claim 26, wherein the method increases the BCVA of the subject relative to a subject that did not receive the triamcinolone drug formulation.

28. The method of claim 26 or 27, wherein the BCVA is assessed using an Early Treatment of Diabetic Retinopathy Study (ETDRS) visual acuity charts protocol.

29. The method of claim 26 or 27, wherein the increase in the BCVA is a gain of about 5, about 6, about 7, about 8, about 9, about 10, about 12, about 14, about 15, about 16, about 17, about 18, about 19, about 20, or more letters.

30. The method of claim 29, wherein the increase in the BCVA is a gain of at least 12 letters.

31. The method of claim 29, wherein the increase in the BCVA is a gain of at least 15 letters.

32. The method of any one of claims 26-31, wherein the increase in BCVA is maintained for at least 36 weeks following the last dose of triamcinolone drug formulation to the SCS.

33. The method of any one of claims 26-31, wherein the increase in BCVA is maintained at for at least 48 weeks following the first dose of triamcinolone drug formulation to the SCS.

34. A method for achieving a durable clinical outcome in a subject having macular edema associated with noninfectious posterior uveitis, noninfectious intermediate uveitis, or noninfectious panuveitis, the method comprising non-surgically administering an effective amount of a triamcinolone drug formulation to the suprachoroidal space (SCS) of the eye of the subject, wherein the durable clinical outcome comprises:
(i) a reduction in retinal thickness in the eye of the subject by at least about 20 μm, at least about 40 μm, at least about 50 μm, at least about 100 μm, at least about 150 μm, or at least about 200 μm; and/or
(ii) an increase in BCVA visual acuity score of the subject relative to a baseline measurement prior to treatment of the subject with triamcinolone drug formulation, wherein the increase in the visual acuity score is a gain of about 5, about 6, about 7, about 8, about 9, about 10, about 12, about 14, about 15, or more letters;
wherein the durable clinical outcome is maintained for at least 36 weeks following the administration of the last dose of the triamcinolone drug formulation.

35. The method of claim 34, wherein the durable clinical outcome is maintained for at least 48 weeks following the administration of the first dose of the triamcinolone drug formulation.

36. The method of claim 34, wherein the increase in the visual acuity score is a gain of at least about 10 letters.

37. The method of claim 34, wherein the increase in the visual acuity score is observed as early as 2 weeks, 3 weeks, 4 weeks, 5 weeks, 6 weeks, 7 weeks, 8 weeks following the first dose of the triamcinolone drug formulation.

38. The method of claim 34, wherein the increase in the visual acuity score is observed as early as 4 weeks following the first dose of the triamcinolone drug formulation.

39. The method of claim 34, wherein the durable clinical outcome comprises a statistically significant difference in the reduction in retinal thickness and/or the increase in the visual acuity score, relative to a subject that did not receive the triamcinolone drug formulation.

40. The method of claim 34, wherein the durable clinical outcome is maintained for at least 48 weeks following the administration of the first dose of the triamcinolone drug formulation.

41. The method of claim 34, wherein the uveitis is intermediate uveitis, and wherein the durable clinical outcome is a reduction in retinal thickness of at least about 100 μm.

42. The method of claim 34, wherein the uveitis is posterior uveitis or panuveitis, and wherein the durable clinical outcome comprises a reduction in retinal thickness of at least about 100 μm and an increase in the visual acuity score of a gain of at least about 10 letters.

43. A method of treating macular edema associated with noninfectious uveitis in a subject in need thereof, the method comprising non-surgically administering an effective amount of a triamcinolone drug formulation to the suprachoroidal space (SCS) of the eye of the human subject in need of treatment, wherein the treatment with the triamcinolone drug formulation is initiated promptly upon diagnosis of the subject with macular edema and/or uveitis.

44. The method of claim 43, wherein the treatment is initiated within 6 months of diagnosis of the noninfectious uveitis.

45. The method of claim 43, wherein the treatment is initiated within 3 months of diagnosis of macular edema in the subject.

46. The method of claim 43, wherein the treatment is initiated within 100 days of diagnosis of the noninfectious uveitis.

47. The method of claim 43, wherein the subject has sudden disease onset prior to treatment.

48. The method of claim 43, wherein the method results in (i) a decrease in retinal thickness relative to a baseline measurement prior to treatment of the subject with the triamcinolone drug formulation; and/or (ii) an increase in a visual acuity score of the subject relative to a baseline measurement prior to treatment of the subject with the triamcinolone drug formulation.

49. The method of claim 43, wherein the method results in a decrease in retina thickness and/or an increase in a visual acuity score, relative to a subject that did not receive the triamcinolone drug formulation.

50. The method of claim 43, wherein the method results in (i) a decrease in retinal thickness or (ii) an increase in a visual acuity score of the subject, relative to a subject that received the triamcinolone drug formulation more than 6 months later than the diagnosis of the noninfectious uveitis.

51. The method of claim 43, wherein the method results in (i) a decrease in retinal thickness or (ii) an increase in a visual acuity score of the subject, relative to a subject that received the triamcinolone drug formulation more than 3 months later than the diagnosis of the macular edema.

52. The method of any one of claims 43-51, wherein the retinal thickness is decreased by at least about 20 μm, at least about 40 μm, at least about 50 μm, at least about 100 μm, at least about 150 μm, or at least about 200 μm.

53. The method of claim 52, wherein the method results in resolution of macular thickening and decreases an average retinal thickness to less than 300 μm.

54. The method of any one of claims 43-53, wherein the decrease in retinal thickness is maintained for at least 36 weeks following the last dose of triamcinolone drug formulation to the SCS.

55. The method of any one of claims 43-54, wherein the decrease in retinal thickness is maintained at for at least 48 weeks following the first dose of triamcinolone drug formulation to the SCS.

56. The method of claim 43, wherein the retinal thickness is decreased by at least about 120 μm at 24 weeks following administration of the first dose of triamcinolone drug formulation to the SCS.

57. The method of claim 43, wherein the retinal thickness is decreased by at least about 170 μm at 48 weeks following administration of the first dose of triamcinolone drug formulation to the SCS.

58. The method of any one of claims 43-57, wherein the increase in the visual acuity score is a Best Corrected Visual Acuity score gain of about 5, about 6, about 7, about 8, about 9, about 10, about 12, about 14, about 15, about 16, about 17, about 18, about 19, about 20, or more letters.

59. The method of any one of claims 43-58, wherein the effective amount of triamcinolone in the drug formulation is about 4 mg.

60. The method of any one of claims 43-59, wherein the method comprises administering two doses of the triamcinolone drug formulation to the SCS of the eye of the subject.

61. The method of claim 60, wherein the two doses of the triamcinolone drug formulation are administered about 1, 2, 3, 4, 5, 6, 7, 8, 9, 10, 11, 12, 13, 14, 15, 16, or 17 weeks apart.

62. The method of claim 60 or 61, wherein the two doses of the triamcinolone drug formulation are administered about 12 weeks apart.

63. The method of any one of claims 1-62, wherein the triamcinolone drug formulation is administered to the subject by suprachoroidal injection comprising the use of an SCS microinjector.

64. The method of claim 63, wherein the SCS microinjector comprises a 30 gauge needle that is about 900 μm or about 1100 μm in length.

65. A method of treating macular edema associated with noninfectious uveitis by achieving a clinical outcome in a subject in need thereof, the method comprising non-surgically administering an effective amount of a triamcinolone drug formulation to the suprachoroidal space (SCS) of the eye of the human subject in need of treatment, wherein the human subject is 50 years old of age or older, and wherein the clinical outcome comprises an increase in BCVA visual acuity score of the subject relative to a baseline measurement prior to treatment of the subject with triamcinolone drug formulation, wherein the increase in the visual acuity score is a gain of about 5, about 6, about 7, about 8, about 9, about 10, about 12, about 14, about 15, or more letters.
